(19) United States
(12) Patent Application Publication
(10) Pub. No.: US 2026/0315368 A1
(43) Pub. Date: Oct. 15, 2026

(54) Cosmetic Composition Comprising a Natural Resin

(71) Applicant: L'OREAL, Paris (FR)

(72) Inventors: Angélina ROUDOT, Chevilly Larue (FR); Lyubov LUKYANOVA, Chevilly Larue (FR); Nathalie GUILLIER, Chevilly Larue (FR); Vanessa OLSZEWSKI, Saint-Ouen (FR)

(73) Assignee: L'OREAL, Paris (FR)

(21) Appl. No.: 18/880,937

(22) PCT Filed: Jul. 5, 2023
(86) PCT No.: PCT/EP2023/068487

§ 371 Date: Jan. 3, 2025

(30) Foreign Application Priority Data
Jul. 5, 2022 (FR) 2206856

(51) Int. Cl. A61K 8/9789; A61K 8/06; A61K 8/34; A61K 8/73; A61K 8/92; A61K 8/9767; A61Q 1/00; A61Q 5/00; A61Q 19/00

(52) U.S. Cl. CPC A61K 8/9789; A61K 8/062; A61K 8/34; A61K 8/731; A61K 8/922; A61K 8/9767; A61Q 1/00; A61Q 5/00; A61Q 19/00; A61K 2800/43; A61K 2800/542

(57) ABSTRACT

The present invention relates to a cosmetic composition comprising, in a physiologically acceptable medium: at least one volatile oil, at least one volatile alcohol, at least one natural resin, and at least one modified polysaccharide; and to the use of such a composition for improving the persistence and/or the resistance to friction without increasing the tacky effect of a film obtained by applying said composition to the keratin materials.

### TECHNICAL FIELD OF THE INVENTION

The present invention relates to a cosmetic composition comprising at least one natural resin, at least one volatile oil, at least one volatile alcohol, and at least one modified polysaccharide in specific weight ratios. The invention also relates to a cosmetic process which implements the application of such a cosmetic composition to keratin materials, particularly human keratin materials such as the skin, the hair or the eyelashes.

### CONTEXT OF THE INVENTION

Cosmetic products often require the use of a film-forming polymer to obtain a deposit of the product on keratin materials that has good cosmetic properties. It is necessary for the film-forming deposit to have good persistence, for the deposit not to transfer during contact with the fingers or clothing, and also good resistance on contact with water, notably rain or during showering and for the deposit to be insensitive to perspiration or sebum, and also to food fats, notably dietary fats such as oils.

Dispersions of particles of polymers, generally acrylic, in organic media such as hydrocarbon-based oils are commonly used as film-forming agents in makeup products such as mascaras, eyeliners, eyeshadows or lipsticks. Silicone-based resins are also used with the aim of improving the cosmetic persistence. These dispersions are not always satisfactory in terms of resistance to fatty substances, particularly dietary fats or sebum, which may be a curb on their use in lip makeup, for example.

In addition, the field of cosmetics formulation is undergoing rapid change. Consumers have expectations of greater use of natural products, the need to be reassured regarding ingredients in cosmetic formulations, in particular regarding their harmlessness, their low environmental footprint, their origin, or else their renewable nature. In recent years, replacing synthetic polymers, and particularly silicones, in cosmetic formulations has become a major challenge.

Although some natural resins, such as rosin (colophony resin) have already been explored as tackifying resins, this is necessarily in combination with a high proportion of silicone resins, for example with a view to increasing the shine and the good persistence of lip makeup (FR2918272). However, document WO2013/147113 discusses several problems encountered with such ingredients, for example: a loss of flexibility of the cosmetic film is often observed as the amount of silicone resin increases; a tacky effect of the cosmetic film appears as the amount of rosin increases. This results in a “heavy”, thick or clumped makeup sensation.

Consumers are used to certain sensory properties and textures, for example characteristic of silicones and synthetic polymer fillers. For this reason, the cosmetic chemist must respond to a dual challenge: on the one hand, that of naturalness, where silicone resins and synthetic polymers are being gradually replaced by more natural starting materials, or starting materials of a more natural origin, and, on the other hand, that of performance and sensoriality in order for more natural formulas to have efficacy and sensory properties which are at least equivalent to those of the less natural formulas they are intended to replace.

Finally, succeeding in formulating natural resins which are solid at ambient temperature (25° C.) in a cosmetic product having a fluid liquid texture remains a technical challenge. Typically, natural resins are said to be soluble in chlorinated solvents or in benzene compounds, or in large quantities of alcohols. Such solvents cannot be considered for a cosmetic use for caring for or making up the skin, in particular the lips, where even ethanol over a certain content is likely to cause discomfort, dryness, irritation or even a skin-burning sensation. Therefore, the present invention also aims to make it possible and simple to prepare cosmetic compositions offering improved persistence, based on ingredients which are as natural as possible, and in a cosmetically acceptable medium.

In particular, the aim of the present invention is to provide a cosmetic composition, the residual film of which after application adheres well to the keratin materials, is elastic and shows the least possible fragmentation and the least possible detachment from the substrate, which is not tacky, has good persistence with respect to external attacking factors such as friction, is resistant to sweat and to sebum, and is relatively insensitive to oils such as dietary oils.

The inventors have shown that, surprisingly a particular combination of volatile alcohol and volatile oil made it possible to effectively solubilize a natural resin, including semi-solid and solid natural resin, at ambient temperature (25° C.), which, combined with a modified polysaccharide, made it possible to obtain a cosmetic composition for which the film obtained after application proves both particularly resistant to friction and non-tacky after drying.

### SUMMARY OF THE INVENTION

The present invention relates to a cosmetic composition (A) comprising, in a physiologically acceptable medium:
a—at least one volatile oil,
b—at least one volatile alcohol,
c—at least one natural resin, and
d—at least one modified polysaccharide.

Preferably, the weight ratio of the total amount of volatile oil(s) and of volatile alcohol(s) to the amount of natural resin(s) is greater than 1.

Preferably, the present invention relates to a cosmetic composition (A) comprising, in a physiologically acceptable medium:
a—at least one volatile oil,
b—at least one volatile alcohol,
c—at least one natural resin, and
d—at least one modified polysaccharide,
wherein:
   the weight ratio of the amount of volatile oil(s) to the amount of natural resin(s) is greater than 0.5, preferably greater than 1;
and/or
   the weight ratio of the amount of volatile alcohol(s) to the amount of natural resin(s) is greater than 0.5, preferably greater than 1.

The present invention also relates to a cosmetic composition comprising, in a physiologically acceptable medium, at least one oily phase of composition (A) as defined above. The oily phase of the invention is preferably continuous.

The present invention also relates to a process for coating keratin materials, more particularly for making up and/or caring for keratin materials, such as the skin, characterized in that it comprises the application, to the keratin materials, of a composition as defined according to the invention.

The present invention further relates to the use of a composition as defined according to the invention for improving the persistence on the skin and/or the resistance to friction of a cosmetic film without increasing the tacky effect of said film obtained by applying said composition to the keratin materials.

### DETAILED DESCRIPTION OF THE INVENTION

For the purposes of the present invention and unless otherwise indicated:

The term “keratin materials” means the skin, mucous membranes and/or skin appendages. Preferably, the keratin materials are the skin, particularly the facial skin, mucous membranes such as the lips, and/or skin appendages such as the eyelashes.

The compositions according to the invention may be cosmetic or dermatological compositions. They are preferably cosmetic compositions.

The composition according to the invention contains a physiologically acceptable medium.

In the present invention, the term “physiologically acceptable medium” means a nontoxic medium that is compatible with keratin materials, particularly with the skin (including the interior of the eyelids), mucous membranes, the hair or the lips of human beings. A cosmetic composition is a product having a pleasant appearance, odour and feel, and intended for topical application.

The term “anhydrous composition” means a composition containing less than 5% by weight of water relative to the total weight of the composition, preferably less than 1% by weight of water, even more preferentially less than 0.5% by weight of water relative to the total weight of the composition, and particularly which is free of water.

The term “volatile substance” means any substance that can evaporate on contact with the skin in less than one hour, at ambient temperature and atmospheric pressure. Said volatile substance is liquid at ambient temperature, particularly has a vapour pressure of greater than or equal to 2.66 Pa, at ambient temperature (25° C.) and atmospheric pressure, preferably within the range from 2.66 Pa to 40 000 Pa, preferably from 2.66 Pa to 13 000 Pa, and preferably from 2.66 Pa to 1300 Pa.

The vapour pressure may be measured according to the static method or via the effusion method by isothermal thermogravimetry, depending on the vapour pressure of the oil (standard OCDE 104).

The term “soluble or solubilized compound” means a compound which can be dissolved in a liquid or which is miscible, and forms just a single homogeneous phase when it is incorporated in the liquid.

The term “natural compound” means a compound which is obtained directly from the earth or soil, notably from plants via, where appropriate, one or more physical processes, especially physical extraction processes, for instance grinding, refining, distillation, purification or filtration.

The term “compound of natural origin” means a natural compound that has undergone one or more additional chemical or industrial treatments, giving rise to modifications that do not affect the essential qualities of this compound and/or a compound predominantly comprising natural constituents that may or may not have undergone transformation(s) as indicated above.

Complying with the principles of green chemistry, this transformation/treatment uses eco-friendly processes and does not involve any substances which harm the environment.

As non-limiting examples of additional chemical and industrial treatments bringing about modifications which do not affect the essential qualities of a natural compound, mention may be made of those permitted by the controlling bodies, such as Ecocert (Reference system for biological and ecological cosmetic products, January 2003), or defined in recognized handbooks in the field, such as “Cosmetics and Toiletries Magazine”, 2005, volume 120, 9:10.

The expressions “of between . . . and . . . ” and “ranging from . . . to . . . ” should be understood as meaning limits included, unless otherwise specified.

The expressions “at least one” and “one or more” are synonymous and can be used interchangeably.

In the description and the examples, unless otherwise indicated, the contents and percentages are percentages by weight. The percentages are thus expressed by weight relative to the total weight of the composition. The ratios are also weight ratios. The temperature is expressed in degrees Celsius, unless otherwise indicated, and the pressure is atmospheric pressure, unless otherwise indicated.

### Composition (A)—Oily Phase

According to a first aspect, a subject of the present invention is a composition as defined previously.

The applicant has observed, surprisingly, that composition A of the invention comprising natural resin(s), particularly dissolved natural resin(s), in a specific mixture of volatile oil(s) and volatile alcohol(s) as defined below, in combination with a modified polysaccharide, made it possible to obtain a film having improved persistence and which is resistant to friction and non-tacky after drying the film.

### Natural Resins

A resin is generally defined as a solid, highly viscous or liquid substance of plant or synthetic origin. Resins have a number of characteristics specific to them, such as:
the ability to permanently harden, for example for the synthetic resins under the influence of temperature and for the natural resins under the influence of oxygen;
their insolubility in water and above all their good tack and adhesive properties. Standard ISO4618:2014 (fr) defines a resin as a “generally amorphous macromolecular product with a consistency ranging from the solid state to the liquid state”.

Natural resins are virtually exclusively of plant origin (fossil or harvested) and are secreted then exuded by plants for roles of defence, protection and communication within their ecosystem. An exception to this is shellac, which is of animal origin and is secreted by the insect Coccus lacca.

For the purposes of the invention, “natural resin” and in particular “plant resin” means any substance comprising a minimal content of terpenic compounds, i.e. at least 30% by weight of terpenic compounds relative to the total weight of the substance (or material) in question, as defined chemically below, said substance being derived directly or indirectly from the secretion and exudation, mainly by plants (and more rarely by animals), of a substance for roles of defence, protection and communication with their ecosystem.

Advantageously, the natural resin according to the invention is insoluble in water at ambient temperature (unlike latices or gums, for example).

Natural resins are also considered to be natural adhesives which have the inherent ability to polymerize consistently and predictably by themselves without synthetic chemistry.

Preferably, the natural resin used in the composition according to the invention has a number-average molecular weight of less than or equal to 10 000 g/mol. The resin preferably has a number-average molecular weight of less than or equal to 10 000 g/mol, particularly ranging from 250 to 10 000 g/mol, preferably less than or equal to 5000 g/mol, particularly ranging from 250 to 5000 g/mol, better still less than or equal to 2000 g/mol, particularly ranging from 250 to 2000 g/mol and even better still less than or equal to 1000 g/mol, particularly ranging from 250 to 1000 g/mol. The number-average molecular weights (Mn) are determined by gel permeation liquid chromatography (THF solvent, calibration curve established with linear polystyrene standards, refractometric detector).

### Thermal Properties

Advantageously, the resins according to the invention are characterized in that they have a softening point, which denotes the temperature of transition from a pseudo-solid state to a plastic state during heating.

Preferably, the resins of the invention have a softening point within the range from 20° C. to 150° C., more preferentially from 30° C. to 100° C., even more preferentially from 40° C. to 90° C.

The softening point is the temperature at which a product reaches a certain degree of softening under standardized conditions. It denotes the temperature of transition from a pseudo-solid state to a plastic state during heating. It can be measured by the ring and ball method (or RBT, ring and ball temperature) for resins according to standard ASTM E284;

Depending on the class thereof, some of the resins according to the invention can also have a melting temperature, preferably of less than 360° C., preferentially less than 190° C., and even more preferentially less than 90° C.

According to a preferred form of the invention, the resins do not have a melting temperature.

The melting point (or melting temperature) of a substance at a given pressure corresponds to the temperature at which the liquid and solid states of this substance can coexist in equilibrium;

Preferably, the resins of the invention have a glass transition temperature preferably within the range from 0° C. to 200° C., more preferentially from 10° C. to 100° C., even more preferentially from 20° C. to 90° C. and even more preferably still from 30° C. to 70° C.

The glass transition temperature (Tg) of a material represents the temperature range through which the material passes from a rubbery state to a vitreous, solid (rigid) state.

The thermal properties, in particular the Mp and Tg of the resins, can be measured by DSC (Differential Scanning calorimetry), for example by means of a DSC 8000 apparatus from Perkin Elmer, according to:
Protocol 1: Determining melting temperature Mp and crystallization temperature Tc: Starting materials alone or solubilized/dispersed in solvents, stainless steel dishes, sweeping from 5° C. to 90° C., sweep rate of 5° C. min-1.
Protocol 2: Determining glass transition temperature Tg: measurement on second heating. Aluminium dishes (40 μl) are used, containing the starting materials, a temperature sweep between −100° C. and 150° C. (with isotherms) is carried out in order to observe the glass transition temperature. The temperature ramp applied is 10° C./min for the glass transition temperatures (2 cycles).

### Botanical Definition of Resins:

Natural resins of plant origin or animal origin are defined conventionally by Ullmann's Encyclopedia of Industrial Chemistry, “Resins, Synthetic” 2012 Wiley-VCH Verlag GmbH & Co. KGaA, Weinheim, DOI: 10.1002/14356007.a23_089.pub2.

Natural resins may be classified by the botanical aspects thereof. The resins can be derived from gymnosperms (uncovered seeds) and angiosperms (covered seeds); the latter are subdivided into monocotyledons (with one embryonic leaf) and dicotyledons (with two embryonic leaves). They can also be selected according to their physical and chemical properties.

Natural resins particularly include rosins (tall oil rosins, wood or gum originating from tree and plant exudates; wood extracts; or by-products from paper manufacturing), fossil resins such as amber; extracted resins such as asphaltite; shellacs such as those produced by insect secretion; and the main derivatives thereof.

The resins of the invention are preferably of plant origin, particularly from plants or trees. Fossil resins are (hard and semi-hard) resins collected from the ground, where ancient forests, long since disappeared, once stood. Some of them are no longer even known with certainty. Some fossil resins have undergone considerable changes in their chemical structure due to ageing or maturation, which may have taken thousands of years. The transition from fossil resins to recent resins can vary. They may for example include resins which are both found in fossilized form and are harvested from living plants. Semi-fossil varieties are collected at the base of the trees which produced them (Ullmann's Encyclopedia of Industrial Chemistry, “Resins, Natural” 2012 Wiley-VCH Verlag Gmbh & Co. KGaA, Weinheim, DOI: 10.1002/14356007.a23_073) (Techniques de I'Ingénieur [Engineering Techniques], “Resines Naturelles” [Natural Resins], 1982 Bernard Delmond).

Harvested resins are recent (tender). They are harvested from plants which are all living. Depending on their composition, they are subdivided into:
oleoresin: natural solution of resin in an essential oil;
balsam: resin characterized by a high proportion of benzoic and cinnamic acids and esters thereof;
gum: resin composed essentially of polysaccharides;
gum-resin: mixture of resins and hydrophilic gums;
latex: milky composition of organic substances dispersed in an aqueous medium: (Techniques de I'Ingénieur [Engineering Techniques], “Résines Naturelles” [Natural Resins], 1982 Bernard Delmond).

Among the resins, in particular the recent resins, of the invention, resins which are soluble in oils and/or alcohols are preferred to water-soluble forms such as latices or gums.

According to a preferred mode of the invention, the resins of the invention are harvested resins; these are particularly beneficial from an ecological perspective, since they are self-renewing.

Preferably, the resins of the invention are recent. Advantageously, the resins used according to the invention rely on resources which are not in competition with those intended for food applications. Advantageously, the resins employed in the compositions of the invention originate from the recycling of co-products of the papermaking industry.

### Chemical Definition of Resins

Chemically, natural resins are complex mixtures of several classes of compounds, the presence and content of which define the glass of the resin (oleoresin, balsam, gum, etc.): essential oils, neutral and acidic constituents and polysaccharides (present exclusively in gums).

The components which characterize resins are the terpenic compounds that they contain, preferably at a content of at least 30% by weight relative to the weight of resin. “Terpenic compounds” means terpenes, hydrocarbons formed from isoprene having the general formula (C5H8)n, and the numerous derivatives thereof (alcohols, aldehydes, ketones, acids, etc.) comprising a terpene structure (Académie de Montpellier. The resins: https://tice.ac-montpellier.fr/ABCDORGA/Famille/Terpenes.html).

Among the terpenic hydrocarbons, a distinction is made between: monoterpenes of empirical formula C10H16 (n=2), sesquiterpenes of empirical formula C15H24 (n=3), diterpenes (C20H32) (n=4), sesterterpenes (C25H40) (n=5), triterpenes (C30H48) (n=6), tetraterpenes (C40H64) (n=8) and other polyterpenes. Some have an acyclic structure; they comprise a number of double bonds which corresponds to their empirical formula: 3 for C10H16; 5 for C20H32; 7 for C30H48. Others have one or more rings, so a reduced number of double bonds, for example for C10H16 one ring and 2 double bonds, or 2 rings and one double bond.

Advantageously, the resins of the invention contain at least 30% of terpenic compounds, preferably at least 40% by weight of terpenic compounds, preferably at least 50% of terpenic compounds, and even more preferably at least 60% of terpenic compounds, or even better still at least 70%, by weight relative to the total weight of resin or of resinous substance used as starting material in the composition according to the invention.

Monoterpenic and sesquiterpenic compounds are predominantly volatile compounds, constituting for example essential oils. Polyterpenic compounds derived from terpenes where n is greater than or equal to 4 (such as derivatives of diterpenes and triterpenes) are resinous compounds of a rather solid nature.

According to a preferred embodiment of the invention, the resins comprise at least 10%, preferably at least 20% by weight, preferably at least 30% by weight, preferably at least 35% by weight, of polyterpenic compounds, i.e. of compounds derived from terpenes where n is greater than or equal to 4, relative to the total weight of resin, representing 100%. Thus, preference is given to resins having a fraction which is solid at ambient temperature (25° C.). Advantageously, said resins used according to the invention are not volatile.

Advantageously, the polyterpenic compounds of the resins or resinous substances used in the composition of the invention are predominantly (to more than 50% by weight relative to the total weight of polyterpenes) derived from diterpenes and/or from triterpenes.

According to a preferred embodiment of the invention, the resins comprise less than 70% by weight of monoterpenic or sesquiterpenic compounds, i.e of compounds derived from terpenes where n is less than 4, relative to the total weight of resin, representing 100%; preferably, said resins comprise less than 60% by weight, preferably less than 50% by weight, preferably less than 30% by weight, preferably less than 15% by weight, of monoterpenic or sesquiterpenic compounds derived from terpenes where n is less than 4 relative to the total weight of resin, representing 100%. Thus, for the compositions of the invention, preference is given to limiting the use of the most volatile resins, since they are less effective in terms of the persistence of a cosmetic film.

A non-exhaustive list of terpenic compounds which may be contained in the natural resins of the invention has been produced. It lists families of terpenic compounds, subdivided on the bases of the characteristic groups (alcohol function, ketone function, acid function, etc.) of each compound (listed below).

### Examples of Monoterpenic Compounds

Advantageously, the monoterpene compounds of the resin are chosen from α-pinene, β-pinene, 3-carene, camphene, dipentene, P-cymene, B-myrcene, α-phellandrene, sabinene, α-thujene, limonene, octyl ethanoate, neryl ethanoate, bornyl ethanoate, geranyl ethanoate, α-terpineol, cineol, linalool, borneol, derivatives thereof and mixtures thereof.

### Examples of Sesquiterpenic Compounds

Advantageously, the sesquiterpene compounds of the resin are chosen from: α-copaene, β-caryophyllene, β-bisabolene, β-gurjunene, alpha-gurjunene, allo-aromadendrene, β-bourbonene, delta-cadinene, α-guaiène, α-elemene, β-elemene, d-elemene, α-copaene, α-selinene, β-selinene, β-bourbonene, lindestrene, furanoeudesma-1,3-diene, α-cubebene, farnesol, α-elemol, viridiflorol, t-cadinol, β-elemol, germacrone, curzerenone, derivatives thereof and mixtures thereof.

### Examples of Diterpenic Compounds

Advantageously, the diterpenic compounds of the resin are chosen from: Abietic acid, pimaric acid, sandaropimaric acid, comunic acid, levopimaric acid, pallustric acid, isopimaric acid, dehydroabietic acid, neoabietic acid, agathic acid, cembrene A, cembrene C, isocembrene, Vercilla-4 (20),7,11-triene, incensole, totarol, sandaracopimarinol, cembrenol, derivatives thereof and mixtures thereof.

### Examples of Triterpenic Compounds

Advantageously, the triterpenic compounds of the resin are chosen from: 3β,20 (S)-dihydroxydammar-24-ene, dammarenolic acid, dammardienone, hydroxydammarenone (I or II), dammarenediol I (or II), dammadienol, 11-keto-β-boswellic acid (KBA), 11-keto-β-boswellic acid acetate (AKBA), β-boswellic acid, ursolic acid, mangiferonic acid, benthamic acid, ursolic aldehyde, α-amyrenone, α-amyrin, β-amyrin, uvaol, oleanolic acid, oleanonic acid, moronic acid, oleanonic aldehyde, acetyl-lupeolic acid, lupeolic acid, lupeol betulonal, hydroxyhopanone, derivatives thereof and mixtures thereof.

According to a first embodiment of the invention, the resin(s) used according to the present invention contain at least one diterpenic compound, preferably derived from abietic acid, which is natural or chemically modified.

Preferably, the diterpenic compound(s), particularly derived from abietic acid, are present within the resin at weight contents of at least 20%, preferably at least 30%, and even more preferentially at least 40%, by weight relative to the total weight of the natural resin.

Mention may particularly be made of colophony resins t) such as rosinates, containing such diterpenic compounds.

According to a second embodiment of the invention, as an alternative or indeed in addition to the first, the resin(s) used according to the present invention contain at least one triterpenic compound, preferably chosen from the following triterpenic compounds: alpha-amyrin, beta-amyrin, alpha-amyrone, beta-amyrone, dammadienone, dammadienol, ursolic aldehyde, hydroxyhopanone, oleanonic aldehyde, ursolic acid, oleanonic acid, oleanolic acid, and mixtures thereof.

The total content of triterpenic compounds, particularly the content of those preferred above, in the resin used according to the invention is advantageously at least 10%, preferably at least 20%, even more preferentially at least 30%, and preferably at least 35% by weight relative to the total weight of the natural resin.

Mention may particularly be made of the frankincense resins k), Protium heptaphyllum or else Shorea robusta, containing such triterpenic compounds.

The chemical composition of a resin may be analysed by conventional techniques known to those skilled in the art, such as gas chromatography GC analysis, chromatographic analysis with flame ionization detection, referred to as GC-FID, or GC/MS analysis, which consists in using a mass spectrometer coupled to a gas chromatograph; preferably by GC-FID.

The following paper mentions these common methods: “Développements méthodologiques en TLC/MALDITOF M S et GC/MS pour l'analyse des composés terpénoïdes présents dans les résines végétales” [Methodological developments in TLC/MALDITOF MS and GC/MS for the analysis of terpenoid compounds present in plant resins], https://tel.archives-ouvertes.fr/tel-01581308.

Definition of the Resins by their Origin:

Advantageously, the natural resin(s) according to the invention are selected from: a) acaroid resins, b) ambers, c) asphaltite and gilsonite, d) Peru balsam, e) Tolu balsam, f) benzoin resins, g) Canada balsam, h) copal resins (particularly kauri copal resins, copal resins from Manilla, West African copals such as Congolese, Angolan or Camaroonian copals, East African copals such as Zanzibari or Madagascan copals, South American copals such as Brazilian or Colombian copals), i) damars, j) elemis, k) frankincenses, l) galbanums, m) labdanums, n) mastics, o) myrrh, p) sandarac, q) shellacs, r) styrax (storax), s) Venice turpentine (larch, turpentine essence), t) rosins, particularly rosin, rosinate and u) tall oils, v) resins extracted from plant waxes, and mixtures of these resins; preferably, the natural resin(s) used according to the invention are chosen from: j), k), t), u) and v), and the mixtures of these resins; preferably, the natural resin(s) are chosen from j), k), and v), and the mixtures of these resins; it being understood that the resin(s) of the invention can be esterified, salified, form adducts, be phenol-modified, and/or dimerized and/or additionally hydrogenated.

### j) Elemis

“Elemis” is a generic term to define the group of recent natural resins derived from plants of the Burseraceae family (Canarium indicum). Each type is described according to its country of origin. According to a particular embodiment of the invention, the elemi resin used originates from the Philippines, particularly Manila elemi. To extract it, the trees are cut and a flow of pathological resin appears, which solidifies over time. The elemis are yellowish to greenish in colour, opaque, similar to a pomade, slimy, tacky and solidify into brownish resins scattered with crystals.

Elemis are soluble in aromatic solvents, in alcohols, esters and carbon disulfide; and less soluble in aliphatic solvents. Elemis have an acid number of between 18 and 34, a saponification number of between 25 and 60, and a softening point of approx. 80. Balsams which exude elemis contain up to 30% of essential oils.

According to a preferred embodiment of the invention, the resin(s) of the invention are chosen from elemis, particularly elemi originating from the Canarium luzonicum family, in pure form or mixed with a latex, for example. Mention may be made of the Canarium luzonicum elemi resin sold under the name ELEMI RESIN.

According to a particular embodiment of the invention, the resin(s) are chosen from j) elemis.

k) Frankincenses (olibanum)

Frankincenses are present in the United Arab Emirates, Oman, Somalia, Ethiopia and Eastern India. Frankincense resins are recent and are taken from the Boswellia carterii tree. Amazonian frankincense resins also exist. The bark is intentionally injured in order to obtain a milky extract which is recovered after drying. Preferably, the resin(s) of the invention are chosen from frankincenses, particularly Amazonian frankincenses.

Frankincense resins are pale yellow and form irregular round or globular beads. They generally contain from 20% to 40% by weight (approx. 33%) of boswellic acid (C32H5204). Frankincenses have an acid number of between 30% and 50% (indirect) and are moderately soluble in ethanol in basic medium.

According to a particular embodiment of the invention, the resin(s) of the invention are chosen from frankincenses, particularly Amazonian frankincense resins sold under the name Protium heptaphyllum resin, or PROTIUM RESIN, or WHITE BREU RESIN, and frankincense resins originating from the sal tree, Shorea robusta.

Advantageously, the resin(s) are in a mixture with one or more fatty substances as defined below according to the invention, preferably chosen from volatile or non-volatile oils. Mention may be made for example of Shorea robusta resin with sunflower seed oil (SHOREA ROBUSTA RESIN, HELIANTHUS ANNUUS (SUNFLOWER) SEED OIL, TOCOPHEROL: 50-75% by weight of Shorea robusta resin, 25-50% by weight of sunflower seed oil) sold under the name KAHLRESIN 6720, and Shorea robusta resin with octyldodecanol (SHOREA ROBUSTA RESIN and OCTYLDODECANOL 50-70% by weight of Shorea robusta resin, 30-50% by weight of octyldodecanol) sold by KAHLWAX. According to a particular embodiment of the invention, the resin(s) are chosen from k) frankincenses.

### t) Rosins

Preferably, the natural resin(s) are chosen from rosins. Rosins are recent resins from renewable resources and can be modified (for example esterified, hydrogenated, substituted).

Rosin gums are preferably purified, distilled, from the balsam of various pine essences (up to 80 different species).

Their composition is determined by the climate, the soil composition and other botanical and meteorological factors. For example, mention may be made of the rosins originating from Pinus austriaca (black pine) Austria, Central America, caribaea (slash pine), United States, Caribbean, densiflora Japan, elliottii United States, halepensis (Aleppo pine) Greece, Portugal, Spain, langifolia India, maritima (seashore pine) France, Spain, Portugal, massoniana (Chinese red pine) China, mercusii Indonesia, Burma, Philippines, nigra (black pine) Austria, oocarpa Central America, Honduras, palustris (swamp pine), United States, (longleaf pine), pseudostrobus Central America, Mexico, sylvestris (Scots pine) Germany, Poland, tonkinensis China, yunnanensis China.

The average composition is approx. 70 to 75% rosin and 20 to 25% turpentine essence. Wood rosin [8050-09-7]

Rosin originates from stumps in the USA which have remained in the ground for at least 10 years in order for the resin-rich duramen to be available.

The pine stumps contain between 10% and 30% by weight (approx. 19%) of rosin, between 1% and 10% by weight (preferably 4%) of turpentine oil, between 1% and 10% by weight (preferably 4%) of resins which are insoluble in petroleum ether, between 20% and 30% by weight (preferably 23%) of water and between 40% and 60% by weight (preferably 50%) of cellulose and of lignin type.

According to a particular embodiment of the invention, the resin(s) are chosen from rosins.

### u) Tall Oil Rosins (Rosin and Rosinate) [8052-10-6]

Tall oil rosins often contain small amounts of higher fatty acids, particularly with a carbon number of greater than or equal to 6 carbon atoms. According to one embodiment, tall oil rosins are free of oxocarboxylic acid. They are particularly soluble in organic solvents. The colophony resins of the invention in particular comprise rosin acids belonging to the terpenes. The numbering of the carbon atoms in the molecules of rosin acid is indicated using abietic acid as an example.

Rosin acids have the molecular chemical formula C₂₀ H₃₀ O₂ and therefore belong to the diterpene family (four isoprene units). A large number of isomers of tricyclic rosin acids exist, which differ in the position of the two double bonds.

Advantageously, said resin according to the invention is chosen from gum rosin, obtained by incision on live trees, wood rosin, which is extracted from pine wood or stumps, and tall oil (“tall oil rosin”), which is obtained from a by-product originating from the production of paper. Advantageously, said resin(s) comprise rosin acids, preferably predominantly chosen from acids of abietic and pimaric type, and particularly chosen from levopimaric acid, neoabietic acid, abietic acid, dehydroabietic acid, tetrahydroabietic acid, dihydroabietic acid, dextropimaric acid, isodextropimaric acid, or else pallustric acid, and mixtures thereof.

The rosin derivatives can result in particular from the polymerization, hydrogenation and/or esterification (for example with polyhydric alcohols, such as ethylene glycol, glycerol or pentaerythritol) of rosin acids. Examples that may be mentioned include the rosin esters sold under the reference Foral 85, Pentalyn H and Staybelite Ester 10 by Hercules; Sylvatac 95 and Zonester 85 by Arizona Chemical, or Unirez 3013 by Union Camp.

According to one embodiment of the invention, the resin(s) are chosen from rosinates (salts of alkaline agents of rosin acids, particularly salts of alkali metals such as sodium or potassium, alkaline-earth metals such as calcium, or metals such as zinc or magnesium).

According to another preferred embodiment of the invention, the resin(s) are chosen from rosin acid esters, particularly esters of rosin acids as defined above and of (C1-C6) alkanols, polyhydroxy (C1-C6)alkane polyols such as glycerol, pentaerythritol, and mixtures thereof, more preferentially chosen from glyceryl rosinate sold under the name RESIESTER GUM A 35, glyceryl rosinate as a mixture with a hydrogenated vegetable oil and/or castor seed oil (GLYCERYL ROSINATE, RICINUS COMMUNIS SEED OIL, HYDROGENATED VEGETABLE OIL sold by EFP BIOTEK) pentaerythrityl rosinate sold under the name RESIESTER N 35 S and RESIESTER 80.

According to another embodiment of the invention, the resin(s) are chosen from poly(carboxy) (C2-C6)alkane or poly(carboxy) (C2-C6)alkene adducts, particularly of maleic acids with rosin acids.

According to another embodiment of the invention, the resin(s) are chosen from phenol-modified rosins. Particularly those modified by (C1-C4)alkylene phenols or diphenols, optionally substituted with one or more (C1-C4)alkyl groups such as methyl or tert-butyl, more particularly rosins modified by 4-tert-butylphenol and 4,4′-isopropylidenediphenol (bisphenol A).

According to another embodiment of the invention, the resin(s) are chosen from dimerized rosins, particularly those in which the abietic acid is polymerized. The rosins preferably contain more than 50% of dimeric acids and are thus referred to as dimerized rosins. According to one embodiment, the rosins are polymerized and contain from 30% to 90% by weight of dimeric acid (particularly at least 40%, 60% or 80% of dimeric acids). According to a preferred embodiment of the invention, the resin(s) are chosen from hydrogenated rosins. The double bonds, particularly of the acids such as abietic acid, are subject to oxidation, which can be eliminated by hydrogenation. Of course, the resin(s) of the invention can be esterified, salified, adducts, phenol-modified, and/or dimerized and additionally hydrogenated.

According to a preferred embodiment, the resin contains at least one ester of rosin acid chosen from the group consisting of glyceryl rosinate, pentaerythrityl rosinate, silicone rosinate, diethylene glycol rosinate, hydrogenated rosinate dilinoleyl dimer, dipentaerythrityl hexahydroxystearate/hexastearate/hexarosinate, glyceryl dibehenate/hydrogenated rosinate, glyceryl diisostearate/hydrogenated rosinate, trihydrogenated glyceryl rosinate, glycol rosinate, hydrogenated methyl rosinate, methyl rosinate, hydrogenated pentaerythrityl rosinate, hydrogenated triethylene glycol rosinate, and mixtures thereof.

According to a particular embodiment, the resin(s) of the invention are chosen from hydrogenated pentaerythrityl rosinate (PENTAERYTHRITYL HYDROGENATED ROSINATE), hydrogenated methyl rosinate (METHYL HYDROGENATED ROSINATE) sold under the name SYMRISE BIO4326.

Moreover, the resin(s) of the invention may be mixed with fatty substances c) as defined below, particularly waxes or butters. Mention may be made of the mixtures of glyceryl rosinate with one or more fatty substances c), particularly chosen from waxes or butters such as the mixture with shea butter or olive oil such as (GLYCERYL ROSINATE, RICINUS COMMUNIS SEED OIL, HYDROGENATED VEGETABLE OIL), BUTYROSPERMUM PARKII (SHEA BUTTER) GLYCERYL ROSINATE, OLEA EUROPAEA (OLIVE) OIL UNSAPONIFIABLES GLYCERYL ROSINATE, OLEA EUROPAEA (OLIVE) OIL UNSAPONIFIABLES, sold by SHEA BUTTER & GLYCERYL ROSINATE & OILS.

v) Resins Extracted from Plant Waxes

Natural plant waxes per se are not considered resins. Although they are among the substances secreted/excreted by plants and naturally contain a very low content of resins, they contain less than 30% by weight of terpenes relative to the total weight of wax. For example, carnauba wax is secreted naturally by the leaves of a palm tree, Copernica cerifera, to prevent the leaves from dehydrating. Candelilla wax is obtained from a shrub named Euphorbia antisyphilitica which originates from northern Mexico. The wax protects the plant from its environment and prevents excessive evaporation. For example, candelilla wax is composed mainly of hydrocarbons (approximately 50%, chains from 29 to 33 carbon atoms), of higher-molecular-weight esters (20% to 29%), of free acids (7% to 9%) and of resins (12-14%, mainly triterpenic esters).

Nevertheless, the definition of “natural resins” for the purposes of the present invention also includes resins resulting from plant waxes, when they have been concentrated, isolated or extracted beforehand from these waxes, as long as the resinous or terpenic ingredient in question contains the minimal content of terpenes (30% by weight relative to the total weight of the ingredient) required by the present invention. Mention may particularly be made of candelilla resin (pure 100% resin, extracted from the corresponding wax) having the INCI name: EUPHORBIA CERIFERA (CANDELLILA) WAX EXTRACT, sold under the name CANDELILLA RESIN E-1 by JAPAN NATURAL PRODUCTS. Document WO2013/147113 A1 also refers to carnauba resin, a terpenic resin extracted from carnauba wax which has similar physical properties to those of the natural resins conventionally described, such as a softening temperature and not a melting temperature, which distinguishes a resin from a wax.

Table 1 of the examples shows some characteristic differences between the waxes and the resins according to the invention, relating to their thermal properties.

The resins have a softening point and a glass transition temperature, but not a melting temperature.

The opposite applies for waxes, which have a melting temperature.

The resin(s) are preferably chosen from resin(s) j), k), and t) as defined previously, and resins v) extracted from waxes, particularly candelilla or carnauba wax and mixtures thereof.

### Resins Preferred According to the Invention:

According to a preferred embodiment of the invention, the resin(s) are chosen from the following references, indicated by their INCI name, used alone or as a mixture:
EUPHORBIA CERIFERA (CANDELILLA) WAX EXTRACT, such as CANDELILLA RESIN E-1 sold by JAPAN NATURAL PRODUCTS, BOTANICAL RESIN sold by CERA RICA NODA, TOWAX-1F 12 sold by TOA KASEI (resin of type v);
PROTIUM HEPTAPHYLLUM RESIN, or PROTIUM RESIN, or WHITE BREU RESIN, which may for example be sold by CITROLEO or Ephyla (resin of type k)
Frankincense resins originating from the sal tree, SHOREA ROBUSTA RESIN. The resin(s) may be as a mixture with one or more fatty substances c) as defined below, preferably chosen from volatile or non-volatile oils. Mention may be made for example of Shorea robusta resin with sunflower seed oil (SHOREA ROBUSTA RESIN, HELIANTHUS ANNUUS (SUNFLOWER) SEED OIL, TOCOPHEROL: 50-75% by weight of Shorea robusta resin, 25-50% by weight of sunflower seed oil) sold under the name KAHLRESIN 6720, and Shorea robusta resin with octyldodecanol (SHOREA ROBUSTA RESIN and OCTYLDODECANOL 50-70% by weight of Shorea robusta resin, 30-50% by weight of octyldodecanol) sold by KAHLWAX (resin of type k)
rosin acid esters such as GLYCERYL ROSINATE sold under the name RESIESTER GUM A 35, glyceryl rosinate as a mixture with a hydrogenated vegetable oil and/or castor seed oil (GLYCERYL ROSINATE, RICINUS COMMUNIS SEED OIL, HYDROGENATED VEGETABLE OIL sold by EFP BIOTEK), pentaerythrityl rosinate sold under the name RESIESTER N 35 S and RESIESTER 80 or hydrogenated rosinates such as hydrogenated pentaerythrityl rosinate (PENTAERYTHRITYL HYDROGENATED ROSINATE), hydrogenated methyl rosinate (METHYL HYDROGENATED ROSINATE) sold under the name SYMRISE BIO4326 (resin of type t).

### Resins Even More Preferred According to the Invention:

According to a more preferred embodiment of the invention, the resin(s) are chosen from the following references, indicated by their INCI name, used alone or as a mixture:
EUPHORBIA CERIFERA (CANDELLILA) WAX EXTRACT, such as CANDELILLA RESIN E-1 sold by JAPAN NATURAL PRODUCTS, BOTANICAL RESIN sold by CERA RICA NODA, TOWAX-1F12 sold by TOA KASEI (resin of type v);
PROTIUM HEPTAPHYLUM RESIN, or PROTIUM RESIN, or WHITE BREU RESIN, which may for example be sold by CITROLEO or Ephyla (resin of type k)
SHOREA ROBUSTA RESIN, Frankincense resins originating from the sal tree. The resin(s) may be as a mixture with one or more fatty substances c) as defined below, preferably chosen from volatile or non-volatile oils. Mention may be made for example of Shorea robusta resin with sunflower seed oil (SHOREA ROBUSTA RESIN, HELIANTHUS ANNUUS (SUNFLOWER) SEED OIL, TOCOPHEROL: 50-75% by weight of Shorea robusta resin, 25-50% by weight of sunflower seed oil) sold under the name KAHLRESIN 6720, and Shorea robusta resin with octyldodecanol (SHOREA ROBUSTA RESIN and OCTYLDODECANOL 50-70% by weight of Shorea robusta resin, 30-50% by weight of octyldodecanol) sold by KAHLWAX (resin of type k).

According to a preferred embodiment of the invention, the resin(s) are chosen from EUPHORBIA CERIFERA (CANDELLILA) WAX EXTRACT.

Advantageously, the resin(s) is (are) present in the composition of the invention at a content with the range from 0.1% to 40%, preferably from 0.5% to 35%, preferably from 1% to 30%, preferably from 1.2% to 25%, preferably from 1.5% to 25%, preferably from 2% to 25%, preferably from 3% to 22%, preferably from 3% to 20%, for example from 5% to 20%, preferably from 3% to 15%, preferably from 3% to 10%, preferably from 3% to 8%, by weight relative to the total weight of the composition, representing 100%.

Advantageously, the composition of the present invention comprises less than 10%, preferably less than 5%, preferably less than 1%, preferably less than 0.5%, preferably less than 0.1%, preferably is free of synthetic resin.

Advantageously, the composition of the present invention comprises less than 10%, preferably less than 5%, preferably less than 1%, preferably less than 0.5%, preferably less than 0.1%, preferably is free of silicone resin, i.e. synthetic resin in which the base structure is a chain comprising siloxane groups (silicon-oxygen-silicon bonds).

### Oils

The composition of the invention comprises an oily phase, advantageously a continuous oily phase, comprising at least one volatile oil chosen from volatile hydrocarbon-based oils, volatile silicone oils and mixtures thereof.

Said phase is liquid (in the absence of structuring agent) at ambient temperature (25° C.) and atmospheric pressure (1.013×10⁵ Pa). It is organic, i.e. comprising at least carbon and hydrogen atoms, and water-immiscible.

The oily phase comprises at least one volatile oil and optionally ingredients which are soluble or miscible in said phase.

The total concentration of oily phase of the composition of the invention is advantageously within the range from 5% to 100%, preferably from 10% to 98%, preferably from 15% to 90% by weight, preferably from 20% to 80% by weight, preferably from 25% to 70% by weight, preferably from 30% to 60% by weight, relative to the total weight of the composition.

The term “oil” denotes a water-immiscible compound which is liquid at 25° C. and atmospheric pressure (1.013×10⁵ Pa).

The term “immiscible” means that the mixing of the same amount of water and oil, after stirring, does not result in a stable solution comprising only a single phase, under the abovementioned temperature and pressure conditions. Observation is performed by eye or using a phase-contrast microscope, if necessary, on 100 g of mixture obtained after sufficient stirring with a Rayneri blender to produce a vortex within the mixture (as a guide, 200 to 1000 rpm), the resulting mixture being left to stand, in a closed flask, for 24 hours at room temperature before observation.

### Volatile Oils

The term “volatile oil” denotes an oil having a vapour pressure of greater than or equal to 1.3 Pa, preferably greater than or equal to 2.66 Pa, at ambient temperature (25° C.) and atmospheric pressure, preferably within the range from 2.66 Pa to 40 000 Pa, preferably from 2.66 Pa to 13 000 Pa, and preferably from 2.66 Pa to 1300 Pa. Advantageously, the volatile oil used in the composition of the present invention has a vapour pressure within the range from 1.3 Pa to 13 000 Pa, preferably from 2.66 Pa to 13 000 Pa, and preferably from 2.66 Pa to 1300 Pa.

In contrast, the term “non-volatile oil” means an oil with a vapour pressure at 25° C. and atmospheric pressure which is non-zero and is less than 2.66 Pa and more particularly less than 0.13 Pa.

By way of example, the vapour pressure may be measured according to the static method or via the effusion method by isothermal thermogravimetry, depending on the vapour pressure of the oil (standard OCDE 104).

The volatile oil(s) are preferably present at a content within the range from 1% to 90% by weight, preferably from 2% to 70%, preferably from 3% to 50%, preferably from 5% to 45% by weight, preferably from 8% to 40% by weight, and even more preferentially from 10% to 35% by weight, relative to the total weight of the composition. The volatile oil(s) are advantageously chosen from volatile hydrocarbon-based oils, volatile silicone oils and mixtures thereof, preferably chosen from volatile hydrocarbon-based oils.

### Volatile Hydrocarbon-Based Oil

The term “hydrocarbon-based oil” means an oil mainly containing carbon and hydrogen atoms and possibly one or more functions chosen from hydroxyl, ester, ether and carboxylic functions. A hydrocarbon-based oil thus consequently does not comprise any silicon or fluorine atoms.

The term “apolar hydrocarbon-based oil” means an hydrocarbon-based oil comprising only carbon and hydrogen atoms, which is preferably non-aromatic (also called a hydrocarbon).

The term “polar hydrocarbon-based oil” denotes hydrocarbon-based oils mainly comprising carbon and hydrogen atoms and one or more functions chosen from hydroxyl, ester, ether and carboxylic functions, i.e. oils having solely C, H and O.

As examples of volatile hydrocarbon-based oils that may be used in the invention, mention may be made of:
hydrocarbon-based oils having from 8 to 16 carbon atoms, and in particular C8-C16 isoalkanes (also referred to as isoparaffins), such as isododecane (also referred to as 2,2,4,4,6-pentamethylheptane), isodecane, isohexadecane and, for example, the oils sold under the trade names Isopar® or Permethyl®,
C6-C16 linear alkanes, for example C11-C15 alkanes, alone or as mixtures, for instance hexane, decane, undecane or tridecane, isoparaffins such as, or n-dodecane (C12) and n-tetradecane (C14) sold by Sasol under the respective references PARAFOL 12-97 and PARAFOL 14-97, the undecane-tridecane mixture, the mixtures of n-undecane (C11) and of n-tridecane (C13) obtained in Examples 1 and 2 of patent application WO 2008/155059 from Cognis, and mixtures thereof, and also mixtures of n-undecane (C11) and of n-tridecane (C13), such as Cetiol Ultimate® from BASF;
volatile, non-aromatic, cyclic C5-C12 alkanes;
branched C8-C16 esters, isohexyl neopentanoate;
short-chain esters (containing from 3 to 8 carbon atoms in total) such as methyl acetate, ethyl acetate, propyl acetate, n-butyl acetate or isobutyl acetate, for example sold by Solvay, Dow or Oxea;
volatile carbonate hydrocarbon-based oils of structure R′1-O—CO—O—R′2 in which R′1 and R′2 independently denote a linear, branched or cyclic C4-C8 alkyl group, preferably a C4-C8 alkyl group. It may be preferable for R1 and R2 to be identical. Preferably, R′1 and R′2 denote a linear butyl alkyl radical, a pentyl group. Advantageously, the ether oil is chosen from dibutyl carbonate and dipentyl carbonate;
volatile ether oils of formula R1OR2 in which R1 and R2 independently denote a linear, branched or cyclic C4-C8 alkyl group, preferably a C4-C8 alkyl group. It may be preferable for R1 and R2 to be identical.

Linear alkyl groups that may be mentioned include a butyl group and a pentyl group. Branched alkyl groups that may be mentioned include a 1-methylpropyl group, a 2-methylpropyl group, a t-butyl group and a 1,1-dimethylpropyl group. Advantageously, the ether oil is chosen from dicaprylyl ether and dicapryl ether, most particularly dicaprylyl ether.

Use may also be made of other volatile hydrocarbon-based oils, such as petroleum distillates, in particular those sold under the name Shell Sol T by Shell; or else volatile linear alkanes, such as those described in patent application DE102008012457 from Cognis.

The volatile hydrocarbon-based oils are preferably chosen from hydrocarbon-based oils of hydrocarbon type (thus apolar hydrocarbon-based oils, consisting solely of carbon and hydrogen) having from 8 to 16 carbon atoms, and mixtures thereof, and particularly:
branched C8-C16 alkanes, such as isoalkanes (also known as isoparaffins), isododecane, isodecane or isohexadecane, and for example the oils sold under the Isopar or Permethyl trade names, alone or as mixtures,
linear alkanes, for example C11-C15 alkanes, alone or as mixtures, and
mixtures thereof.

The volatile hydrocarbon-based oil(s) are particularly chosen from C6-C16 alkanes and in particular alkanes such as dodecane, tetradecane, isohexadecane, mixtures of undecane and tridecane, and isoparaffins such as C13-C16 isoparaffin.

According to a preferred embodiment of the invention, the volatile oil(s) are linear or branched hydrocarbon-based oils, which are volatile, particularly chosen from undecane, decane, dodecane, isododecane, isohexadecane, tridecane, tetradecane and a mixture thereof, preferably comprising isododecane and/or a mixture of undecane and tridecane. According to a particular embodiment of the invention, the volatile oil(s) of the invention are a mixture of C9-C12 alkanes, preferably of natural origin, the chains of which comprise from 9 to 12 carbon atoms, preferably linear or branched C9-C12 alkanes. This mixture is particularly known under the INCI name C9-C12 ALKANE, CAS 68608-12-8, VEGELIGHT SILK® sold by BioSynthls.

According to a preferred embodiment, the volatile oil(s) are at least partially of plant origin.

### Volatile Silicone Oil

The term “silicone oil” refers to an oil comprising at least one silicon atom, and particularly at least one Si—O group, and more particularly an organopolysiloxane. The volatile silicone oils may be chosen from linear, branched or cyclic silicone oils, such as polydimethylsiloxanes (PDMSs) containing from 3 to 7 silicon atoms.

Examples of such oils that may be mentioned include octyl trimethicone, hexyl trimethicone, methyl trimethicone, decamethylcyclopentasiloxane, octamethylcyclotetrasiloxane, dodecamethylcyclohexasiloxane, decamethyltetrasiloxane, polydimethylsiloxanes such as those sold under the reference DC 200 (1.5 cSt), or DC 200 (3 cSt) by Dow Corning or KF 96 A from Shin-Etsu; alone or as mixtures.

According to a specific form of the invention, use will be made of a mixture of at least one volatile hydrocarbon-based oil and of at least one volatile silicone oil, and more particularly of a mixture of isododecane and of dodecamethylpentasiloxane.

Advantageously, the composition according to the invention contains less than 30%, preferably less than 20%, preferably less than 10%, preferably less than 1%, preferably less than 0.5%, preferably less than 0.2%, preferably less than 0.1% by weight of silicone oil, especially of volatile silicone oil, relative to the total weight of the composition, and ideally the composition of the invention is free of any silicone oil.

Preferably, in the composition according to the invention, the volatile oil(s) are chosen from volatile hydrocarbon-based oils.

The composition of the present invention preferably comprises isododecane, C9-C12 linear or branched alkanes and/or mixtures of n-undecane (C11) and of n-tridecane (C13); preferably it comprises isododecane.

Preferably, the weight ratio of the amount of volatile oil(s) to the amount of natural resin(s) is within the range from 0.5 to 50, preferably 1 to 30, preferably 3 to 20, preferably 5 to 18, preferably 8 to 15.

According to one embodiment of the invention, the composition can additionally comprise one or more non-volatile oils.

### Non-Volatile Oils

The term “non-volatile oil” means an oil, the vapour pressure of which at 25° C. and atmospheric pressure is non-zero and is less than 2.66 Pa and more particularly less than 0.13 Pa. By way of example, the vapour pressure may be measured according to the static method or via the effusion method by isothermal thermogravimetry, depending on the vapour pressure of the oil (standard OCDE 104).

The non-volatile oil(s) of the invention are of natural or synthetic origin, preferably natural. According to a particular embodiment of the invention, composition C1 or C1′ comprises one or more non-volatile oils.

Among the non-volatile oils, mention may be made of:

### Non-Volatile Silicone Oils

The non-volatile silicone oil may particularly be chosen from the non-volatile silicones having the following INCI names: dimethicone, dimethiconol, trimethyl pentaphenyl trisiloxane, tetramethyl tetraphenyl trisiloxane, diphenyl dimethicone, trimethylsiloxyphenyl dimethicone, phenyl trimethicone, diphenylsiloxyphenyl trimethicone; and also mixtures thereof.

These products are notably sold under the names PH-1555 HRI Cosmetic Fluid (trimethyl pentaphenyl trisiloxane) and Dow Corning 556 Cosmetic Grade Fluid (phenyl trimethicone) by Dow Corning; diphenyl dimethicones such as the products KF-54, KF54HV, KF-50-300CS, KF-53 d and KF-50-100CS or Diphenylsiloxy Phenyl Trimethicone KF56 A sold by Shin-Etsu; the products Belsil PDM 1000 and Belsil PDM 20 sold by Wacker Chemie (trimethylsiloxy phenyl dimethicone), alone or as mixtures.

### Non-Volatile Fluoro Oils

The term “fluoro oil” denotes an oil comprising at least one fluorine atom. The fluoro oil can particularly be chosen from fluorinated polyethers, and also from the fluorosilicone oils and the fluoro silicones as described in EP-A-847752.

### Apolar Non-Volatile Hydrocarbon-Based Oils

The apolar non-volatile hydrocarbon-based oils can be chosen from linear or branched compounds of mineral or synthetic origin, for example:
liquid paraffin,
squalane, such as the reference NEOSSANCE SQUALANE sold by AMYRIS,
isoeicosane,
saturated linear hydrocarbons and mixtures thereof, more particularly of C15-C28, such as the mixtures with for example the following INCI names: C15-19 Alkane, C18-C21 Alkane, C21-C28 Alkane, for example the products Gemseal 40, Gemseal 60, Gemseal 120 sold by Total, Emogreen L19, Emogreen L15 sold by SEPPIC,
hydrogenated or non-hydrogenated polybutenes, for example products of the Indopol range sold by Ineos Oligomers, products having the INCI name HYDROGENATED POLYISOBUTENE
hydrogenated or non-hydrogenated polyisobutenes, for example non-volatile compounds of the Parleam® range sold by NIPPON OIL FATS,
hydrogenated or non-hydrogenated polydecenes, for example non-volatile compounds of the PURESYN® range sold by ExxonMobil,
decene/butene copolymers and butene/isobutene copolymers,
and mixtures thereof.

### Polar Non-Volatile Hydrocarbon-Based Oils

They may be chosen from:
saturated or unsaturated, linear or branched C10-C26 fatty alcohols, preferably monoalcohols. Advantageously, the C10-C26 alcohols are fatty alcohols, which are preferably branched when they comprise at least 16 carbon atoms. Preferably, the fatty alcohol comprises from 10 to 24 carbon atoms, and more preferentially from 12 to 22 carbon atoms, notably such as lauryl alcohol, isostearyl alcohol, oleyl alcohol, 2-butyloctanol, 2-undecylpentadecanol, 2-hexyldecyl alcohol, isocetyl alcohol, octyldodecanol and mixtures thereof;
triglycerides consisting of esters of fatty acids and of glycerol, the fatty acids of which may in particular have chain lengths ranging from C4 to C36, and particularly from C8 to C36, preferably from C18 to C36, it being possible for these oils to be linear or branched, and saturated or unsaturated. By way of example, mention may particularly be made of heptanoic or octanoic triglycerides, caprylic/capric acid triglycerides; plant oils such as wheatgerm oil, sunflower oil, grapeseed oil, sesame seed oil, corn oil, apricot kernel oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passionflower oil, musk rose oil; the liquid fraction of shea butter, and the liquid fraction of cocoa butter; and also mixtures thereof;
linear aliphatic hydrocarbon-based esters of formula RCOOR′ in which RCOO represents a carboxylic acid residue including from 2 to 40 carbon atoms and R′ represents a hydrocarbon-based chain containing from 1 to 40 carbon atoms, aliphatic hydrocarbon-based esters of alkylene glycol, in particular ethylene glycol or propylene glycol, the total number of carbon atoms advantageously being at least 10. As examples of such esters, mention may be made of isoamyl laurate, cetostearyl octanoate, isopropyl myristate, isopropyl palmitate, isopropyl stearate or isostearate, ethyl palmitate, 2-ethylhexyl palmitate, isostearyl isostearate, octyl stearate, isostearyl heptanoate, cocoyl caprylate/caprate, octanoates, decanoates or ricinoleates of alcohols or of polyalcohols, such as propylene glycol dioctanoate, cetyl octanoate, tridecyl octanoate, 2-ethylhexyl palmitate, alkyl benzoate, polyethylene glycol diheptanoate, propylene glycol bis(2-ethylhexanoate) and mixtures thereof, hexyl laurate, neopentanoic acid esters, such as isodecyl neopentanoate, isotridecyl neopentanoate, isostearyl neopentanoate or 2-octyldodecyl neopentanoate, isononanoic acid esters, such as isononyl isononanoate, isotridecyl isononanoate or octyl isononanoate, oleyl erucate, isopropyl lauroyl sarcosinate, diisopropyl sebacate, isocetyl stearate, isodecyl neopentanoate, isostearyl behenate or myristyl myristate;
hydroxylated esters, such as polyglyceryl-2 triisostearate;
aromatic esters such as tridecyl trimellitate, C12-C15 alcohol benzoate, the 2-phenylethyl ester of benzoic acid, and butyloctyl salicylate;
esters of linear fatty acids having a total carbon number ranging from 35 to 70, such as pentaerythrityl tetrapelargonate;
esters of branched C24-C28 fatty acids or fatty alcohols, such as triisoarachidyl citrate, pentaerythrityl tetraisononanoate, glyceryl triisostearate, glyceryl tris(2-decyltetradecanoate), pentaerythrityl tetraisostearate, polyglyceryl-2 tetraisostearate or pentaerythrityl tetra(2-decyltetradecanoate);
the polyesters obtained by condensation of dimer and/or trimer of unsaturated fatty acid and of diol, such as those having the INCI name Dilinoleic Acid/Butanediol Copolymer or Dilinoleic Acid/Propanediol Copolymer; the polyesters obtained by condensation of fatty acid dimer and of diol dimer, such as dimer dilinoleyl dimer dilinoleate;
synthetic ethers containing from 10 to 40 carbon atoms, such as dicaprylyl ether;
dialkyl carbonates, the two alkyl chains possibly being identical or different, such as dicaprylyl carbonate;
vinylpyrrolidone copolymers such as vinylpyrrolidone/1-hexadecene copolymer (INCI name); and
mixtures thereof.

According to one embodiment, the non-volatile oil(s) are chosen from non-volatile silicone oils, non-volatile hydrocarbon-based oils, polar hydrocarbon-based oils as defined previously, and mixtures thereof preferably chosen from non-volatile hydrocarbon-based oils, polar hydrocarbon-based oils as defined previously and mixtures thereof.

According to one embodiment, the non-volatile hydrocarbon-based oil(s) comprise or consist of at least one non-volatile oil chosen from linear aliphatic hydrocarbon-based esters of formula RCOOR′ in which RCOO represents a carboxylic acid residue containing from 2 to 40 carbon atoms, and R′ represents a hydrocarbon-based chain containing from 1 to 40 carbon atoms, aliphatic hydrocarbon-based esters of alkylene glycol, in particular ethylene glycol or propylene glycol as defined previously, more preferentially chosen from isoamyl laurate, isopropyl myristate, isodecyl neopentanoate, isostearyl neopentanoate, isononyl isononanoate, cocoyl caprylate/caprate and mixtures thereof, and better still denote isononyl isononanoate.

According to one embodiment, the non-volatile hydrocarbon-based oil(s) comprise or consist of at least one non-volatile oil chosen from saturated or unsaturated, linear or branched C10-C26 fatty alcohols, preferably monoalcohols, which are preferably branched when they comprise at least 16 carbon atoms as described previously, in particular chosen from oleyl alcohol, 2-hexyldecyl alcohol, isocetyl alcohol, octyldodecanol such as the reference Eutanol G sold by BASF, and mixtures thereof. According to one embodiment, the non-volatile hydrocarbon-based oil(s) comprise or consist of at least one non-volatile oil chosen from triglycerides consisting of esters of fatty acids and of glycerol, the fatty acids of which may in particular have chain lengths ranging from C4 to C36, and particularly from C18 C8 to C36, it being possible for these oils to be linear or branched, and saturated or unsaturated as described previously, preferably chosen from heptanoic or octanoic triglycerides, caprylic/capric acid triglycerides and mixtures thereof, and more preferentially caprylic/capric acid triglycerides such as the reference Palmester 3585 sold by KLK Oleo.

According to one embodiment, the non-volatile hydrocarbon-based oil(s) comprise or consist of at least one non-volatile oil chosen from apolar hydrocarbon-based non-volatile oils as described previously, preferably chosen from mixtures of linear, saturated hydrocarbons, more particularly C15-C28 hydrocarbons, hydrogenated or non-hydrogenated polybutenes, and mixtures thereof.

According to one embodiment, the non-volatile hydrocarbon-based oil(s) comprise or consist of at least one non-volatile oil chosen from apolar hydrocarbon-based non-volatile oils chosen from the mixtures whose INCI names are, for example, the following: C15-C19 Alkane, C18-C21 Alkane, C21-C28 Alkane, for example the products Gemseal 40, Gemseal 60, Gemseal 120 sold by Total, Emogreen L19 sold by SEPPIC, Emogreen L15 sold by SEPPIC, the products having the INCI name Hydrogenated Polyisobutene, and mixtures thereof.

According to a particular embodiment of the invention, the non-volatile hydrocarbon-based oil(s) c) comprise or consist of at least one non-volatile oil chosen from isoamyl laurate, isopropyl myristate, isodecyl neopentanoate, isostearyl neopentanoate, isononyl isononanoate, oleyl alcohol, 2-hexyldecyl alcohol, isocetyl alcohol, octyldodecanol, caprylic/capric acid triglycerides, mixtures whose INCI names are, for example, the following: C15-C19 Alkane, C18-C21 Alkane, C21-C28 Alkane, for instance Gemseal 40, Gemseal, products having the INCI name Hydrogenated Polyisobutene, and mixtures thereof, more particularly chosen from products having the INCI name Hydrogenated Polyisobutene, mixtures having the INCI name C15-C19 Alkane, such as Emogreen L15 sold by SEPPIC, and isononyl isononanoate.

According to one embodiment, the non-volatile hydrocarbon-based oil(s) consist of one or more non-volatile polar or apolar hydrocarbon-based oil(s) as defined previously.

According to one embodiment, the non-volatile oil(s) comprise at least one silicone oil as defined above, preferably chosen from dimethicones, such as the grade Belsil DM 5 Plus Dimethicone sold by Wacker, the reference Dowsil SH 200 C Fluid 10 CST sold by Dow Chemical or the reference Xiameter PMX-200 Silicone Fluid 1000 CST sold by Dow Chemical, or Phenyl Trimethicone such as the reference Dowsil SH 556 Fluid sold by Dow Chemical.

Preferably, when the non-volatile oil(s) are a mixture of at least one non-volatile hydrocarbon-based oil preferably chosen from apolar non-volatile hydrocarbon-based oils and polar non-volatile hydrocarbon-based oils with at least one volatile silicone oil, the amount of silicone oil is less than 30%, preferably less than 20%, preferably less than 10%, relative to the total weight of the composition.

According to a particular embodiment of the invention, the optional non-volatile oil(s) are present in the composition in an amount of from 0.1% to 50%, preferably from 0.2% to 40%, preferably from 0.5% to 35%, preferably from 1% to 30%, more preferentially between 2% and 20% by weight relative to the total weight of the composition.

Weight ratio, denoted R, means the ratio of the sum of the masses of volatile oil(s) (VO) to the sum of the masses of non-volatile oils (NVO), defined by:

R=[sum of masses of VO]/[sum of masses of NVO].

Preferably, R is such that 0<R≤10 000, more particularly 0.01<R≤1000; more particularly 0.05<R≤500; preferably, 0.1<R≤100, or even 0.5<R≤50.

The addition of non-volatile oils tends to decrease the resistance of the film to wear, so it is preferable to use them rather at low contents compared to that of the volatile oils, in order to retain optimal persistence while having a comfortable skin sensation (soft, moisturizing and non-tacky sensations after applying a composition).

### Volatile Alcohols

The composition according to the invention comprises at least one volatile alcohol. The term “alcohol” means any chemical compound comprising at least one hydroxyl function in its structure.

The term “volatile substance” means any substance that can evaporate on contact with the skin in less than one hour, at ambient temperature and atmospheric pressure. The volatile substance is liquid at ambient temperature, particularly having a non-zero vapour pressure, at ambient temperature (25° C.) and atmospheric pressure, in particular having a vapour pressure ranging from 0.13 Pa to 40 000 Pa (10-3 to 300 mmHg), and preferably ranging from 1.3 Pa to 13 000 Pa (0.01 to 100 mmHg).

The term “volatile alcohol” means any compound containing at least one hydroxyl group and in which more than 95% by weight of the compound is capable of evaporating in less than one hour at ambient temperature (25° C.) and atmospheric pressure (760 mmHg) on contact with a keratin material such as the skin or the hair, particularly which is capable of evaporating on contact with the skin in less than one hour at ambient temperature and atmospheric pressure. Said volatile substance is liquid at ambient temperature, and preferably has a vapour pressure of greater than or equal to 2.66 Pa, at ambient temperature (25° C.) and atmospheric pressure, preferably within the range from 2.66 Pa to 40 000 Pa, preferably from 2.66 Pa to 13 000 Pa, and preferably from 2.66 Pa to 8000 Pa.

Advantageously, according to the definition of the volatiles substances used in the composition of the present invention, the volatile oil or the volatile alcohol, preferably each of them, has a vapour pressure, at ambient temperature (25° C.) and atmospheric pressure, of greater than or equal to 1.3 Pa, preferably within the range from 1.3 Pa to 13 000 Pa, preferably greater than or equal to 2.66 Pa, preferably within the range from 2.66 Pa to 13 000 Pa.

The vapour pressure may be measured according to the static method or via the effusion method by isothermal thermogravimetry, depending on the vapour pressure of the oil (standard OCDE 104).

The volatile alcohol(s) in accordance with the present invention are preferably chosen from lower C1-C5 alcohols, may be chosen from methanol, ethanol, propanol, isopropanol, n-butanol, isobutanol, tert-butanol, preferably chosen from C1-C4 alcohols, preferably chosen from: ethanol, isopropanol, tert-butanol, n-butanol, and mixtures thereof, more particularly ethanol.

Their viscosity at 20° C., measured with a Haake RheoStress 600 machine, with a rotor 60 mm in diameter, an angle of 2° and a shear rate of 200 s-1, is preferably from 0.3 to 3 mPa·s.

Advantageously, the volatile alcohol(s) are present at contents within the range from 1% to 70% and more preferentially from 5% to 50%, and more particularly from 5% to 30% by weight relative to the total weight of the composition. An advantage of the composition of the invention is being able to limit the content of volatile alcohol(s), which is often the cause of discomfort (dryness, tingling) without losing any solubilizing power.

Preferably, the weight ratio of the amount of volatile alcohol(s) to the amount of natural resin(s) is within the range from 0.5 to 50, preferably 1 to 30, preferably 1.2 to 20, preferably 1.5 to 15.

Preferably, the weight ratio of the amount of volatile oil(s) to the amount of volatile alcohol(s) is within the range preferably from 0.01 to 100, preferably from 0.1 to 10, preferably from 0.25 to 8, preferably from 0.3 to 7.5, preferably from 0.5 to 7,5, preferably from 0.5 to 5, preferably from 0.7 to 5, preferably from 1 to 4, preferably from 1.5 to 4. Preferably, the weight ratio of the amount of volatile oil(s) to the amount of volatile alcohol(s) is greater than 0.25, preferably greater than 0.3, preferably greater than 0.4, preferably greater than 0.5, preferably greater than 0.6, preferably greater than 0.7, preferably greater than 0.9, preferably greater than 1.

Advantageously, in the composition according to the invention, the weight content of volatile oil(s) is greater than the weight content of volatile alcohol(s), which is itself greater than the weight content of natural resin(s), relative to the total weight of the composition (oil >alcohol >resin).

Thus, in the composition according to the invention, the sums(S) of the respective weight contents of volatile oil(s), of volatile alcohol(s) and of natural resin(s) preferably follow the rule:
S (volatile oil(s))>S (volatile alcohol(s))>S (natural resin(s)). The undesired properties of said volatile alcohols are counterbalanced by a carefully selected volatile oil content, while ensuring a sufficient content of volatile alcohol(s) in order to enable the natural resin to be solubilized.

### Modified Polysaccharides

The composition of the invention comprises one or more modified polysaccharides.
a “sugar” is a monosaccharide or polysaccharide radical, and the O-protected sugar derivatives thereof such as sugar esters of (C1-C6)alkylcarboxylic acids such as acetic acid, sugars containing amine group(s) and (C1-C4)alkyl derivatives, such as methyl derivatives, for instance methylglucose. Sugar radicals that may be mentioned include: sucrose, glucose, galactose, ribose, fucose, maltose, fructose, mannose, arabinose, xylose, lactose;
the term “monosaccharide” refers to a monosaccharide sugar comprising at least 5 carbon atoms of formula Cx(H2O)x with x an integer greater than or equal to 5, preferably x is greater than or equal to 6, in particular x is between 5 and 7 inclusive, preferably x=6; they may be of D or L configuration, and of alpha or beta anomer, and also the salts thereof and the solvates thereof such as hydrates;
the term “polysaccharide” refers to a polysaccharide sugar which is a polymer constituted of several saccharides bonded together by O-glycosidic bonds, said polymers being constituted of monosaccharide units as defined previously, said monosaccharide units comprising at least 5 carbon atoms, preferably 6; in particular, the monosaccharide units are linked together via a 1,4 or 1,6 bond as α (alpha) or β (beta) anomer, it being possible for each saccharide unit to be of L or D configuration, and also the salts thereof and the solvates thereof such as the hydrates of said monosaccharides; more particularly, they are polymers formed from a certain number of saccharides (or monosaccharides) having the general formula: —[Cx(H2O)y)]w- or —[(CH2O)x]w-, where x is an integer greater than or equal to 5, preferably x is greater than or equal to 6, in particular x is between 5 and 7 inclusive and preferably x=6, and y is an integer which represents x−1, and w is an integer greater than or equal to 2, particularly between 3 and 3000 inclusive, more particularly between 5 and 2500, preferentially between 10 and 2300, particularly between 15 and 1000 inclusive, more particularly between 20 and 500, preferentially between 25 and 200;

Preferably, the polysaccharide(s) are thickening polymers.

The term “thickening polymer” means a polymer which, when introduced at 1% by weight in an alcoholic or lipoalcoholic solution containing 50% ethanol, or in an oil chosen from liquid petroleum jelly, isopropyl myristate, octyldodecanol or cyclopentadimethylsiloxane, makes it possible to achieve a viscosity of at least 100 cps and preferably of at least 500 cps, at 25° C. and at a shear rate of 1 s⁻¹. This viscosity may be measured using a cone/plate viscometer (Haake R600 rheometer or the like).

The polysaccharide(s) that are useful in the invention are cationic, nonionic, anionic or amphoteric polymers, preferably cationic, nonionic or anionic polymers, better still nonionic polymers, modified by the presence of at least one hydrocarbon-based aliphatic chain, which is cyclic or acyclic, linear or branched, saturated or unsaturated, aromatic or non-aromatic, comprising from 2 to 30 carbon atoms, optionally substituted with one or more atoms or groups a), f), g), h), i), j), l) as defined below and/or p) (di)alkylamino and/or optionally interrupted with one or more heteroatoms or groups a′) to c′) as defined below: i) linear or branched (C5-C28)alkyl, ii) linear or branched (C5-C28)alkenyl, iii) linear or branched (C5-C28)alkynyl, preferably the hydrocarbon-based group is linear; a) halogen such as chlorine or bromine, f) (thio) carboxamide —C(O)—N(Ra)2 or —C(S)—N(Ra)2, g) cyano, h) iso (thio) cyanate, i) (hetero) aryl such as phenyl or furyl, and j) (hetero)cycloalkyl such as anhydride, epoxide or dithiolane, l) R—X with R representing a group chosen from a) cycloalkyl such as cyclohexyl, β) heterocycloalkyl such as sugar, preferably monosaccharide such as glucose, y) (hetero) aryl such as phenyl, 8) cosmetic active agent, m) thiosulfate, and X representing a′) O, S, N(Ra) or Si(Rb)(Rc), b′) S(O)ᵣ, or (thio) carbonyl, c′) or combinations of a′) with b′) such as (thio) ester, (thio)amide, (thio) urea or sulfonamide; Ra representing a hydrogen atom, or a (C1-C4)alkyl group, or aryl(C1-C4)alkyl groups such as benzyl, preferably Ra represents a hydrogen atom; Rb and Rc, which may be identical or different, represent a (C1-C4)alkyl or (C1-C4)alkoxy group, particularly only one substituent; and/or a′) heteroatoms such as O, S, N(Ra), and Si(Rb)(Rc), b′) S(O) r, (thio) carbonyl, c′) or the combinations of a′) with b′) such as (thio) ester, (thio)amide, (thio) urea or sulfonamide with r being equal to 1 or 2, Ra being as defined previously, preferably Ra represents a hydrogen atom, Rb and Rc, being as defined previously

The “polysaccharides” are as defined previously, in addition, the saccharide units-[Cₓ(H₂O)_{y})]_{w}— or —[(CH₂O)ₓ]_{w}— are optionally modified by substitution, oxidation, dehydration, and/or reduction.

As saccharide units of the polysaccharide(s) that are useful in the invention, mention may preferably be made of glucose, galactose, arabinose, rhamnose, mannose, xylose, fucose, anhydrogalactose, galacturonic acid, glucuronic acid, mannuronic acid, galactose sulfate, anhydrogalactose sulfate and fructose.

Mention may notably be made of modified polysaccharides of native gums such as those derived from tree or shrub exudates, algae, seeds or tubers, fungi, bacteria, animal organisms or plants, which have been modified physically or by chemical or enzymatic reactions.

The native gums may be chosen from:
acacia gum (branched polysaccharide of galactose, arabinose, rhamnose and glucuronic acid);
gum ghatti (polymer derived from arabinose, galactose, mannose, xylose and glucuronic acid);
gum karaya (polymer derived from galacturonic acid, galactose, rhamnose and glucuronic acid);
gum tragacanth (polymer of galacturonic acid, galactose, fucose, xylose and arabinose);
agar (polymer derived from galactose and anhydrogalactose);
alginates (polymers of mannuronic acid and glucuronic acid);
carrageenans and furcellerans (polymers of galactose sulfate and anhydrogalactose sulfate);
guar gum (polymer of mannose and galactose);
locust bean gum (polymer of mannose and galactose);
fenugreek gum (polymer of mannose and galactose);
tamarind gum (polymer of galactose, xylose and glucose);
konjac gum (polymer of glucose and mannose);
xanthan gum (polymer of glucose, mannose acetate, mannose/pyruvic acid and glucuronic acid) or dehydroxanthan gum;
gellan gum (polymer of partially acylated glucose, rhamnose and glucuronic acid);
scleroglucan gum (glucose polymer);
cellulose (glucose polymer);
starch (glucose polymer);
inulin and
pectin.

In particular, the modified polysaccharides are derived from: i) acacia gum; ii) gum ghatti; iii) gum karaya; iv) gum tragacanth; v) agar; vi) alginates; vii) carrageenans and furcellerans; viii) guar gum; ix) locust bean gum; x) fenugreek gum; xi) tamarind gum; xii) konjac gum; xiii) xanthan gum or dehydroxanthan gum; xiv) gellan gum; xv) scleroglucan gum; xvi) cellulose; xvii) starch; xviii) inulin; and xix) pectin; preferably chosen from xvi), xvii) and xviii), more preferentially xvii).

The starch molecules xvii) used in the present invention may have as botanical origin cereal plants or else tubers. Thus, the starches are chosen, for example, from the starches of corn, rice, manioc, barley, potato, wheat, sorghum and pea.

The starches may be chemically or physically modified: notably by means of one or more of the following reactions: pregelatinization, oxidation, crosslinking, esterification, etherification, amidation, heat treatments.

According to one embodiment of the invention, the modified polysaccharide(s) are nonionic.

These polymers may be chemically or physically modified. A physical treatment that may notably be mentioned is temperature.

As chemical treatments, mention may be made of esterification, etherification, amidation, oxidation, metathesis and addition reactions. These treatments make it possible to produce polymers that may notably be nonionic, anionic or amphoteric.

Preferably, these chemical or physical treatments are applied to guar gums, locust bean gums, starches and celluloses.

The modifiable starch molecules that may be used to manufacture modified starches according to the present invention may have as botanical origin cereal plants or else tubers. Thus, the starches are for example chosen from the starches of corn, rice, manioc, barley, potato, wheat, sorghum and pea.

The starches may be modified chemically or physically: notably by one or more of the following reactions: pregelatinization, oxidation, crosslinking, esterification, etherification, amidation, heat treatments.

The starch molecules xvii) may be derived from any plant source of starch, notably such as corn, potato, oats, rice, tapioca, sorghum, barley or wheat, which have been modified to link at least one hydrocarbon-based aliphatic chain, which is cyclic or acyclic, linear or branched, saturated or unsaturated, aromatic or non-aromatic, comprising from 6 to 30 carbon atoms optionally substituted with one or more atoms or groups a), f), g), h), i), j), l) as defined above; and/or p) (di)alkylamino and/or optionally interrupted with one or more heteroatoms or groups a′) to c′) as defined above. Use may also be made of the hydrolysates of the abovementioned starches. The modified starch is preferably derived from potato starch.

According to one embodiment, the modified polysaccharides are polysaccharide ethers called alkyl polysaccharides, in which the alkyl radical comprises between 2 and 30, preferably between 2 and 10, more preferentially between 2 and 6 carbon atoms.

Preferably, the alkyl polysaccharides b) according to the invention are derived from cellulose or guar or mixtures thereof.

According to one embodiment, the modified polysaccharides are alkylcelluloses in which the linear or branched alkyl residue comprises between 1 and 10 carbon atoms, in particular between 2 and 6 carbon atoms, preferably between 2 and 3 carbon atoms.

Alkylcellulose is an alkyl ether of cellulose comprising a chain constituted of β-anhydroglucose units linked together by acetal bonds. Each anhydroglucose unit has three replaceable hydroxyl groups, it being possible for all or some of these hydroxyl groups to react according to the following reaction:

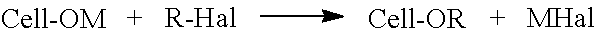

with Hal representing a halogen such as Cl with M representing a cationic counterion such as an alkali metal Na or K, or an alkaline-earth metal, preferably an alkali metal such as Na, Cell. representing a polysaccharide radical such as cellulose, where R represents a linear or branched alkyl group comprising from 1 to 10 carbon atoms, preferably between 2 and 3 carbon atoms, such as methyl or ethyl, and MHal represents the salt generated such as sodium chloride.

Advantageously, the alkylcellulose is chosen from ethylcellulose and propylcellulose. According to a particularly preferred embodiment, the alkylcellulose is ethylcellulose. It is an ethyl ether of cellulose.

Total substitution of the three hydroxyl groups would lead for each anhydroglucose unit to a degree of substitution of 3, in other words to a content of alkoxy groups of between 40% and 60%, notably about 55% (54.88%).

The ethylcellulose polymers used in a composition according to the invention are preferentially polymers having a degree of substitution with ethoxy groups ranging from 2.5 to 2.6 per anhydroglucose unit, in other words comprising a content of ethoxy groups ranging from 44% to 50%.

According to a particular embodiment of the invention, the modified polysaccharide of the invention is an ethylcellulose in powder form. It is, for example, sold under the trade names Ethocel Standard from Dow Chemicals, notably Ethocel Standard 7 FP Premium and Ethocel Standard 100 FP Premium. Other commercially available products, such as those sold by Ashland, Inc. under the names Aqualon Ethylcellulose type-K, type-N and type-T, preferably type-N, such as N7, N100, are particularly suitable for performing the invention.

According to another embodiment, the polysaccharide ethers are alkyl guars i.e. guar gums viii) modified by replacing the hydrogen of hydroxyl with a linear or branched alkyl group, comprising between 1 and 10 carbon atoms, in particular between 2 and 6 carbon atoms, preferably between 2 and 3 carbon atoms such as 2 carbon atoms.

The alkyl guar polymer used in a composition C1 or C′1 according to the invention is preferentially ethylguar.

Ethylguar is known under the INCI name: C₁-C₅ alkyl galactomannan.

More particularly, it has a degree of substitution of 2 to 3, and notably 2.5 to 2.8. Alkylated guar gums (with C₁-C₆ alkyl group), including ethyl guar, are notably described in patent application EP 708114 and document RD9537807 (October 1995), along with their preparation process.

According to one embodiment, the modified polysaccharides b) are polysaccharide esters, in particular esters obtained by reaction between at least one polysaccharide such as dextrin with at least one saturated or unsaturated acid which is linear or branched and including from 2 to 30 carbon atoms, in particular from 10 to 30 carbon atoms.

According to a particular embodiment, the modified polysaccharides of the invention are chosen from xvi) cellulose or derivatives thereof such as hydroxy(C₁-C₅)alkylcelluloses, xvii) starch and xviii) inulin; said polysaccharides xvi), xvii) and xviii) including at least one C₈-C₃₀ fatty chain, such as alkyls, arylalkyls, alkylaryls or mixtures thereof where the alkyl groups are linear or branched, preferably linear, C₈-C₃₀ alkyl groups and in particular:

According to a particular embodiment of the invention, the modified polysaccharide(s) are chosen from saccharide or polysaccharide monoalkyl or polyalkyl esters.

Among the saccharide or polysaccharide monoalkyl or polyalkyl esters that are suitable for use in the invention, mention may be made of dextrin or inulin alkyl or polyalkyl esters. It may notably be a monoester or polyester of dextrin (dextrin being derived from starch xvii) and of at least one fatty acid (such as R—C(O)—OH) and notably corresponding to formula (XVIII) below:

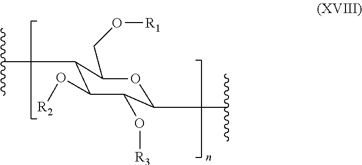

in which formula (XVIII):
   n is an integer greater than or equal to 2, preferably ranging from 3 to 200, notably ranging from 20 to 150, and in particular ranging from 25 to 50,
   R₁, R₂ and R₃, which may be identical or different, are chosen from hydrogen or an acyl group (R—C(O)—) in which the radical R is a linear or branched, saturated or unsaturated hydrocarbon-based group containing from 7 to 29, in particular from 7 to 21, notably from 11 to 19, more particularly from 13 to 17, or even 15, carbon atoms, it being understood that at least one of said radicals R₁, R₂ or R₃ is other than hydrogen.

In particular, R₁, R₂ and R₃ represent a hydrogen atom or an acyl group (R—C(O)—) in which R is a hydrocarbon-based radical as defined previously, with the proviso that at least two of said radicals R₁, R₂ or R₃ are other than hydrogen.

All the radicals R₁, R₂ and R₃ may represent an identical or different acyl group (R—C(O)), and the acyl groups are notably identical.

In particular, n described previously advantageously ranges from 25 to 50, and is notably equal to 38 in the general formula of the saccharide ester that may be used in the present invention.

Notably when the radicals R₁, R₂ and/or R₃, which may be identical or different, represent an acyl group (R—C(O)), derived from a fatty carboxylic acid R—C(O) OH preferably chosen from caprylic, capric, lauric, myristic, palmitic, stearic, arachic, behenic, isobutyric, isovaleric, 2-ethylbutyric, ethylmethylacetic, isoheptanoic, 2-ethylhexanoic, isononanoic, isodecanoic, isotridecanoic, isomyristic, isopalmitic, isostearic, isoaracic, isohexanoic, decenoic, dodecenoic, tetradecenoic, myristoleic, hexadecenoic, palmitoleic, oleic, elaidic, asclepinic, gondoleic, eicosenoic, sorbic, linoleic, linolenic, punicic, stearidonic, arachidonic or stearolic acid, and mixtures thereof.

Preferably, at least one dextrin palmitate is used as fatty acid ester of dextrin. This ester may be used alone or as a mixture with other esters.

Advantageously, the fatty acid ester of dextrin has a degree of substitution of less than or equal to 2.5 based on a glucose unit, notably ranging from 1.5 to 2.5, preferably from 2 to 2.5. The weight-average molecular weight of the dextrin ester may be in particular from 10 000 to 150 000, notably from 12 000 to 100 000 and even from 15 000 to 80 000.

Preferably, the modified polysaccharide(s) of the invention are dextrin esters, and are preferably dextrin palmitates.

Dextrin esters, in particular dextrin palmitates, are commercially available under the name Rheopearl KL2®, MKL2®, TL® or KL® from the company Chiba Flour.

According to one embodiment, the modified polysaccharide is a modified dextrin, preferably a dextrin ester, more particularly a saturated or unsaturated, linear or branched C₁₂-C₂₄ fatty acid ester of dextrin.

Preferably, the dextrin ester is chosen from esters of saturated or unsaturated, linear or branched C₁₄-C₂₄ fatty acids such as myristic acid, palmitic acid or a mixture thereof.

According to one embodiment, the dextrin ester is chosen from dextrin palmitate such as Rheopearl KL2® and Rheopearl TL2® sold by Chiba Flour, dextrin myristate such as the product sold under the reference Rheopearl MKL2® by Chiba Flour, dextrin palmitate/ethylhexanoate sold under the reference Rheopearl TT2®, dextrin palmitate/hexyldecanoate sold under the reference Rheopearl WX, or mixtures thereof. According to a preferred embodiment, the modified polysaccharide denotes dextrin palmitate.

According to one embodiment, the modified polysaccharide is a modified inulin, preferably an inulin ester, more particularly an ester of inulin and of a saturated or unsaturated, linear or branched C₁₂-C₂₄ fatty acid.

Preferably, the inulin ester is chosen from esters of saturated or unsaturated, linear or branched C₁₄-C₂₄ fatty acids such as myristic acid, palmitic acid, stearic acid, preferably stearic acid, and mixtures thereof.

According to one embodiment, the inulin ester is a stearoyl inulin such as the references Rheopearl ISK2® and Rheopearl ISL2® sold by Chiba Flour or mixtures thereof.

According to one embodiment, the modified polysaccharide is a modified cellulose, preferably a cellulose ester, more particularly an ester of cellulose and of a saturated or unsaturated, linear or branched C₂-C₂₄ acid.

Preferably, the cellulose ester is chosen from esters of saturated or unsaturated, linear or branched C₂-C₁₀ acids, preferably C₂-C₆ acids, notably C₂-C₄ acids, such as acetic acid, butyric acid or a mixture thereof.

According to one embodiment, the cellulose ester is a cellulose acetate butyrate such as the reference Eastman Cellulose Acetate Butyrate® sold by Eastman Chemical.

Among the polysaccharide esters, mention may also be made of pullulan esters. Pullulan is a polysaccharide consisting of maltotriose units.

According to one embodiment, the modified polysaccharides are polysaccharide esters. The term “polysaccharide esters” means polysaccharides in which at least one of the hydroxyl radicals is esterified with an acid to form-O—C(O)—R or —C(O)—OR ester groups in which R denotes a saturated or unsaturated radical of 2 to 30 carbon atoms, notably 11 to 19 carbon atoms, preferably 12 to 17 carbon atoms such as 13 carbon atoms. Advantageously, the polysaccharide ester is myristoyl pullulan.

According to another embodiment, the modified polysaccharide(s) of the invention are cationic. Preferably these chemical or physical treatments to obtain at least one cationic group are applied to guar gums, locust bean gums, starches and celluloses.

The cationic groups may be of the primary, secondary, tertiary or quaternary amine type, preferably quaternary, and include a C₆-C₃₀ aliphatic chain.

According to a particular embodiment of the invention, the modified polysaccharide(s) are chosen from quaternized (poly) hydroxyethylcelluloses modified with groups containing at least one aliphatic chain (or fatty chain), such as alkyl, arylalkyl, alkylaryl groups containing at least 8 carbon atoms, or mixtures thereof. The alkyl radicals borne by the quaternized celluloses or hydroxyethylcelluloses preferably include from 8 to 30 carbon atoms. The aryl radicals preferably denote phenyl, benzyl, naphthyl or anthryl groups. Examples of quaternized alkylhydroxyethylcelluloses bearing C₈-C₃₀ fatty chains are the Quatrisoft LM 200R, Quatrisoft LM-X 529-18-AR, Quatrisoft LM-X 529-18-BR (C12 alkyl) and Quatrisoft LM-X 529-8® (C18 alkyl) products sold by the company Dow Corning, the Crodacel QM®, Crodacel QL® (C12 alkyl) and Crodacel QS® (C18 alkyl) products sold by the company Croda and the Softcat SL 100® product sold by the company Dow Corning.

The nonionic guar gums that may be used according to the invention may be modified with C₁-C₂₀ (poly)hydroxylalkylammonium groups, preferably C₁-C₆ (poly) hydroxyalkyl groups; mention may notably be made, by way of example, of hydroxymethyltrimmonium, hydroxyethyltrimmonium, hydroxypropyltrimmonium and hydroxybutyltrimmonium halide groups, preferably hydroxypropyltrimonium halide, preferably chloride.

Such cationic guar gums modified with hydroxyalkylammonium groups are sold, for example, by the company Solvay under the trade names Cationic Jaguar® C-14S Guar Hydroxypropyltrimonium Chloride F, Jaguar® C-13S Guar Hydroxypropyltrimonium Chloride F, Jaguar® C-17 Guar Hydroxypropyltrimonium Chloride, Jaguar® Excel Guar Hydroxypropyltrimonium Chloride, Jaguar® C-500 STD Guar Hydroxypropyltrimonium Chloride, Jaguar® C-162 Hydroxypropyl Guar Hydroxypropyltrimonium Chloride, Jaguar® Optima Guar Hydroxypropyltrimonium Chloride and Jaguar® LS Hydroxypropyl Guar Hydroxypropyltrimonium Chloride.

Preferably, the modified polysaccharide(s) is (are) chosen from:
polysaccharide ethers called alkyl polysaccharides, in which the alkyl radical comprises between 2 and 30, preferably between 2 and 10, more preferentially between 2 and 6 carbon atoms; preferably derived from cellulose or guar or mixtures thereof; preferably the modified polysaccharides are alkylcelluloses in which the linear or branched alkyl residue comprises between 1 and 10 carbon atoms, in particular between 2 and 6 carbon atoms, preferably between 2 and 3 carbon atoms; preferably chosen from ethylcellulose and propylcellulose, preferably the alkylcellulose is ethylcellulose; and
polysaccharide esters; and mixtures thereof.

Preferably, the modified polysaccharide(s) is (are) chosen from: dextrin palmitate, myristoyl pullulan, ethylcellulose, ethyl guar, and mixtures thereof.

The total amount of the modified polysaccharide(s) present in the composition according to the invention is within the range of from 0.05% to 20% by weight, more preferentially from 0.1% to 15% by weight, even more preferably from 0.2% to 12% by weight, and according to a particularly preferred mode from 0.5% to 10% by weight relative to the total weight of the composition.

Preferably, the weight ratio between the total amount of resin(s) and the total amount of modified polysaccharide(s), present in the composition, ranges from 0.05 to 200, more preferentially from 0.1 to 100, more preferentially from 0.2 to 50, or better still from 0.5 to 40, preferably from 0.5 to 20, preferably from 1 to 10.

### Other Optional Components of the Oily Phase

### The Wax(es)

According to a particular embodiment, the composition of the invention comprises one or more waxes.

The term “wax” means a lipophilic compound that is solid at room temperature (25° C.), with a reversible solid/liquid change in state, having a melting point of greater than or equal to 30° C., which may be up to 200° C. and especially up to 120° C.

In particular, the wax(es) that are suitable for use in the invention may have a melting point of greater than or equal to 45° C. and in particular of greater than or equal to 55° C. The composition according to the invention preferably comprises a content of wax(es) ranging from 0.2% to 30% by weight relative to the total weight of the composition, in particular from 0.5% to 20% and more particularly from 1% to 15%.

According to a particular form of the invention, the composition of the invention is solid, in particular anhydrous. It can thus be in the form of a stick.

### The Pasty Compound(s)

According to a particular embodiment, the composition of the invention comprises one or more pasty compounds.

For the purposes of the present invention, the term “pasty compound” means a lipophilic fatty compound with a reversible solid/liquid change of state, having in the solid state an anisotropic crystal organization, and comprising at a temperature of 23° C. a liquid fraction and a solid fraction.

According to a particular embodiment of the invention, the composition according to the invention comprises one or more volatile oils, one or more non-volatile oils, optionally water and optionally one or more organic solvents other than the oils a) and alcohols b) defined according to the invention.

Advantageously, the total content of oily phase is within the range from 5% to 100%, preferably from 10% to 98% by weight, preferably from 20% to 95% by weight, preferably from 30% to 60% by weight, relative to the total weight of the composition.

According to an embodiment of the invention, the composition comprises at least one continuous oily phase having a composition as defined above.

According to a first embodiment, the composition of the invention is in the form of an oily composition, particularly an anhydrous oily composition, such as an oily dispersion or an oily solution.

According to a second embodiment of the invention, the composition additionally has an aqueous phase.

Advantageously, the total content of oily phase is within the range from 5% to 100%, preferably from 10% to 98% by weight, preferably from 20% to 90% by weight, preferably from 30% to 80% by weight, relative to the total weight of the composition.

### Aqueous Phase

The aqueous phase comprises water and optionally water-soluble or water-miscible ingredients, such as water-soluble solvents.

A water suitable for the invention can be a floral water, such as cornflower water, and/or a mineral water, such as Vittel water, Lucas water or La Roche-Posay water, and/or a thermal water.

In the present invention, the term “water-soluble solvent” denotes a compound which is liquid at ambient temperature and miscible with water (miscibility in water of greater than 50% by weight at 25° C. and atmospheric pressure).

The water-soluble solvents that can be used in the composition of the invention may also be volatile.

Mention may in particular be made, among the water-soluble solvents which can be used in the composition in accordance with the invention, of lower monoalcohols having from 1 to 5 carbon atoms, such as ethanol and isopropanol, glycols having from 2 to 8 carbon atoms, such as ethylene glycol, propylene glycol, 1,3-butylene glycol, propanediol, pentylene glycol, glycerol and dipropylene glycol, C₃-C₄ ketones and C₂-C₄ aldehydes. The aqueous phase is preferably present in a concentration of 2% to 95% by weight, preferably from 10% to 90% by weight, preferably within the range from 20% to 80% by weight, more particularly from 30% to 60% by weight, relative to the total weight of said composition.

Surfactants According to a particular embodiment of the invention, the composition also comprises one or more surfactant(s), preferably nonionic or ionic surfactants, or mixtures thereof. According to another particular embodiment of the invention, the composition does not comprise any surfactant.

The term “surfactant” means a compound which modifies the surface tension between two surfaces. The surfactant(s) are amphiphilic molecules, which have two parts of different polarity, one part being lipophilic (which retains fatty substances) which is apolar, the other hydrophilic part (miscible or soluble in water) being polar. The lipophilic part is generally a fatty chain, and the other water-miscible part is polar, and/or protic. The term “ionic” means anionic, cationic, amphoteric or zwitterionic.

The term “fatty chain” means a linear or branched, saturated or unsaturated hydrocarbon-based chain comprising more than 6 atoms, preferably between 6 and 30 carbon atoms and preferably from 8 to 24 carbon atoms.

Emulsifying surfactants are characterized by the value of their HLB (Hydrophilic Lipophilic Balance), the HLB being the ratio of the hydrophilic part to the lipophilic part in the molecule. The term “HLB” is well known to those skilled in the art and is described, for example, in “The HLB system. A time-saving guide to Emulsifier Selection (published by ICI Americas Inc; 1984). For emulsifying surfactants, the HLB generally ranges from 3 to 8 for the preparation of W/O emulsions. The HLB of the surfactant(s) used according to the invention can be determined by the Griffin method or the Davies method. According to a first particular embodiment, the composition of the invention contains at least one silicone or non-silicone nonionic surfactant.

Among the nonionic surfactants according to the invention, mention may be made, alone or as mixtures, of fatty alcohols, α-diols and alkylphenols, these three types of compound being polyethoxylated, polypropoxylated and/or polyglycerolated and containing a fatty chain including, for example, 8 to 22 carbon atoms, the number of ethylene oxide or propylene oxide groups possibly ranging in particular from 2 to 50 and the number of glycerol groups possibly ranging in particular from 2 to 30. Mention may also be made of ethylene oxide and propylene oxide copolymers, condensates of ethylene oxide and of propylene oxide with fatty alcohols; polyethoxylated fatty amides preferably containing from 2 to 30 mol of ethylene oxide, polyglycerolated fatty amides including on average 1 to 5, and in particular 1.5 to 4, glycerol groups, oxyethylenated fatty acid esters of sorbitan containing from 2 to 30 mol of ethylene oxide, fatty acid esters of sucrose, fatty acid esters of polyethylene glycol, alkylpolyglycosides, N-alkylglucamine derivatives, amine oxides such as (C10-C14)alkylamine oxides or N-acylaminopropylmorpholine oxides.

The surfactant(s) represent in total particularly from 0.01% to 30% by weight, preferably from 0.5% to 15% by weight, even more preferentially from 1% to 10% by weight and better still between 1% and 5% by weight of the composition, relative to the total weight of the composition.

### Pigments

According to a particular mode of the invention, the composition additionally comprises at least one pigment.

The term “pigments” means white or coloured, mineral or organic particles, which are insoluble in an aqueous medium, and which are intended to colour and/or opacify the resulting composition and/or deposit. These pigments may be white or coloured, and mineral and/or organic.

Preferably, the composition comprises at least 2% of pigments, preferably at least 5% by weight of pigment(s), more preferentially from 5% to 40% by weight of pigment(s), in particular from 10% to 30% by weight of pigment(s) and preferably from 9% to 20% by weight of pigment(s) relative to the total weight of said composition.

According to a specific embodiment, the pigments used according to the invention are chosen from mineral pigments.

The term “mineral pigment” means any pigment that satisfies the definition in Ullmann's encyclopedia in the chapter on inorganic pigments. Mention may be made, among the mineral pigments of use in the present invention, of zirconium or cerium oxides, and also zinc, iron (black, yellow or red) or chromium oxides, manganese violet, ultramarine blue, chromium hydrate and ferric blue, titanium dioxide, or metal powders, such as aluminium powder and copper powder. The following mineral pigments may also be used: Ta₂O₅, Ti₃O₅, Ti₂O₃, TiO, ZrO₂ as a mixture with TiO₂, ZrO₂, Nb₂O₅, CeO₂ or ZnS.

The size of the pigment of use in the context of the present invention is generally greater than 100 nm and can range up to 10 μm, preferably from 200 nm to 5 μm and more preferentially from 300 nm to 1 μm.

According to a particular form of the invention, the pigments have a size characterized by a D[50] greater than 100 nm and possibly ranging up to 10 μm, preferably from 200 nm to 5 μm and more preferentially from 300 nm to 1 μm.

The sizes are measured by static light scattering using a commercial particle size analyser of MasterSizer 3000® type from Malvern, making it possible to determine the particle size distribution of all of the particles over a wide range which can extend from 0.01 μm to 1000 μm. The data are processed on the basis of the standard Mie scattering theory. This theory is the most suitable for size distributions ranging from submicron to multimicron; it allows an effective particle diameter to be determined. This theory is described in particular in the publication by Van de Hulst, H. C., Light Scattering by Small Particles, Chapters 9 and 10, Wiley, New York, 1957.

D[50] represents the maximum size which 50% by volume of the particles have. In the context of the present invention, the mineral pigments are more particularly iron oxide and/or titanium dioxide. Mention may more particularly be made, by way of examples, of titanium dioxides and iron oxides, which are coated with aluminium stearoyl glutamate, for example sold under the reference NAI® by MIYOSHI KASEI.

As mineral pigments that may be used in the invention, mention may also be made of nacres.

The term “nacres” should be understood as meaning coloured particles of any form, which may or may not be iridescent, notably produced by certain molluscs in their shell, or alternatively synthesized, and which have a colour effect via optical interference.

The nacres may be chosen from nacreous pigments such as titanium mica coated with an iron oxide, titanium mica coated with bismuth oxychloride, titanium mica coated with chromium oxide, titanium mica coated with an organic dye and also nacreous pigments based on bismuth oxychloride. They may also be mica particles, at the surface of which are superposed at least two successive layers of metal oxides and/or of organic dyestuffs.

Examples of nacres that may also be mentioned include natural mica covered with titanium oxide, with iron oxide, with natural pigment or with bismuth oxychloride.

Among the commercially available nacres, mention may be made of the nacres Timica®, Flamenco® and Duochrome® (based on mica) sold by Engelhard, the Timiron® nacres sold by Merck, the Prestige® mica-based nacres sold by Eckart, and the Sunshine® synthetic mica-based nacres sold by Sun Chemical.

The nacres can more particularly have a yellow, pink, red, bronze, orangey, brown, gold and/or coppery colour or tint.

As illustrations of nacres that may be used in the context of the present invention, mention may be made of gold-coloured nacres sold in particular by Engelhard under the name Brilliant gold 212G® (Timica), Gold 222C® (Cloisonne), Sparkle Gold® (Timica), Gold 4504® (Chromalite) and Monarch gold 233X® (Cloisonne); the bronze nacres sold in particular by Merck under the names Bronze Fine® (17384) (Colorona) and Bronze® (17353) (Colorona) and by Engelhard under the name Super bronze (Cloisonne); the orange nacres sold in particular by Engelhard under the names Orange 363C® (Cloisonne) and Orange MCR 101® (Cosmica) and by Merck under the names Passion Orange® (Colorona) and Matte orange (17449)® (Microna); the brown-tinted nacres sold in particular by Engelhard under the names Nu-antique copper 340XB® (Cloisonne) and Brown CL4509® (Chromalite); the nacres with a copper tint sold in particular by Engelhard under the name Copper 340A® (Timica); the nacres with a red tint sold in particular by Merck under the name Sienna Fine® (17386) (Colorona); the nacres with a yellow tint sold in particular by Engelhard under the name Yellow (4502)® (Chromalite); the red-tinted nacres with a golden tint sold in particular by Engelhard under the name Sunstone G012® (Gemtone); the pink nacres sold in particular by Engelhard under the name Tan opal G005® (Gemtone); the black nacres with a golden tint sold in particular by Engelhard under the name Nu antique bronze 240 AB® (Timica); the blue nacres sold in particular by Merck under the name Matte Blue® (17433) (Microna); the white nacres with a silvery tint sold in particular by Merck under the name Xirona Silver®; and the golden-green pinkish-orange nacres sold in particular by Merck under the name Indian Summer® (Xirona), and mixtures thereof.

Mention may also be made, among the pigments that may be used according to the invention, of those having an optical effect different from a simple conventional colouring effect, that is to say a unified and stabilized effect such as is produced by conventional dyestuffs, such as, for example, monochromatic pigments. For the purposes of the invention, the term “stabilized” means devoid of effect of variability of the colour with the angle of observation or also in response to a temperature change.

For example, this material can be chosen from particles with a metallic glint, goniochromatic colouring agents, diffractive pigments, thermochromic agents, optical brighteners, and also fibres, in particular interference fibres. Of course, these various materials can be combined so as to provide the simultaneous display of two effects, indeed even of a novel effect in accordance with the invention.

The particles with a metallic tint that are usable in the invention are in particular chosen from:
particles of at least one metal and/or of at least one metal derivative,
particles comprising a monomaterial or multimaterial organic or inorganic substrate, at least partially covered with at least one layer with a metallic glint comprising at least one metal and/or at least one metal derivative, and
the mixtures of said particles.

Among the metals that may be present in said particles, mention may be made, for example, of Ag, Au, Cu, Al, Ni, Sn, Mg, Cr, Mo, Ti, Zr, Pt, Va, Rb, W, Zn, Ge, Te and Se, and mixtures or alloys thereof. Ag, Au, Cu, Al, Zn, Ni, Mo, Cr and mixtures or alloys thereof (for example, bronzes and brasses) are preferred metals.

The term “metal derivatives” denotes compounds derived from metals, in particular oxides, fluorides, chlorides and sulfides.

Illustrations of these particles that may be mentioned include aluminium particles, such as those sold under the names Starbrite 1200 EAC® by Silberline and Metalure® by Eckart.

Mention may also be made of metal powders of copper or of alloy mixtures such as the references 2844 sold by the company Radium Bronze, metallic pigments, for instance aluminium or bronze, such as those sold under the names Rotosafe 700® from Eckart, silica-coated aluminium particles sold under the name Visionaire Bright Silver® from Eckart, and metal alloy particles, for instance the silica-coated bronze (alloy of copper and zinc) powders sold under the name Visionaire Bright Natural Gold® from Eckart. They may also be particles comprising a glass substrate, such as those sold by the company Nippon Sheet Glass under the names Microglass Metashine®.

The goniochromatic colouring agent may be chosen, for example, from multilayer interference structures and liquid-crystal colouring agents.

Examples of symmetrical multilayer interference structures that may be used in the compositions prepared in accordance with the invention are, for example, the following structures: Al/SiO₂/Al/SiO₂/Al, pigments having this structure being sold by DuPont de Nemours; Cr/MgF₂/Al/MgF₂/Cr, pigments having this structure being sold under the name Chromaflair® by Flex; MoS₂/SiO₂/Al/SiO₂/MoS₂; Fe₂O₃/SiO₂/Al/SiO₂/Fe₂O₃, and Fe₂O₃/SiO₂/Fe₂O₃/SiO₂/Fe₂O₃, pigments having these structures being sold under the name Sicopearl® by BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name Xirona® by Merck (Darmstadt). By way of example, these pigments may be the pigments of silica/titanium oxide/tin oxide structure sold under the name Xirona Magic® by Merck, the pigments of silica/brown iron oxide structure sold under the name Xirona Indian Summer® by Merck and the pigments of silica/titanium oxide/mica/tin oxide structure sold under the name Xirona Caribbean Blue® by Merck. Mention may also be made of the Infinite Colors pigments from Shiseido. Different effects are obtained according to the thickness and the nature of the various layers. Thus, with the Fe₂O₃/SiO₂/Al/SiO₂/Fe₂O₃ structure, the colour changes from greenish gold to reddish grey for SiO₂ layers of 320 to 350 nm; from red to gold for SiO₂ layers of 380 to 400 nm; from violet to green for SiO₂ layers of 410 to 420 nm; from copper to red for SiO₂ layers of 430 to 440 nm.

As examples of pigments with a polymeric multilayer structure, mention may be made of those sold by 3M under the name Color Glitter®.

Use may be made, as liquid crystal goniochromatic particles, for example, of those sold by Chenix and of those sold under the name Helicone® HC by Wacker.

### Hydrophobic Coated Pigments

According to a specific form of the invention, the compositions according to the invention comprise at least one pigment coated with at least one lipophilic or hydrophobic compound and in particular as described in detail below.

This type of pigment is particularly advantageous insofar as it may be considered in a large amount together with a large amount of water. What is more, insofar as they are treated with a hydrophobic compound, they show a predominant affinity for the oily gelled phase, which can then convey them.

Of course, the compositions according to the invention can in parallel contain uncoated pigments.

The coating may also comprise at least one additional non-lipophilic compound.

For the purposes of the invention, the “coating” of a pigment according to the invention generally denotes the total or partial surface treatment of the pigment with a surface agent, absorbed on, adsorbed on or grafted to said pigment.

The surface-treated pigments can be prepared according to surface treatment techniques of chemical, electronic, mechanochemical or mechanical nature which are well known to those skilled in the art. Commercial products can also be used.

The surface agent can be absorbed on, adsorbed on or grafted to the pigments by solvent evaporation, chemical reaction and creation of a covalent bond.

According to one variant, the surface treatment consists of coating the pigments. The coating may represent from 0.1% to 20% by weight and in particular from 0.5% to 5% by weight relative to the total weight of the coated pigment.

The coating may be produced, for example, by adsorption of a liquid surface agent onto the surface of the solid particles by simple mixing with stirring of the particles and of said surface agent, optionally with heating, prior to the incorporation of the particles into the other ingredients of the makeup or care composition.

The coating may be produced, for example, by chemical reaction of a surface agent with the surface of the solid pigment particles and creation of a covalent bond between the surface agent and the particles. This method is particularly described in patent U.S. Pat. No. 4,578,266.

The chemical surface treatment may consist in diluting the surface agent in a volatile solvent, dispersing the pigments in this mixture and then slowly evaporating off the volatile solvent, so that the surface agent is deposited on the surface of the pigments.

### Lipophilic or Hydrophobic Treatment Agent

When the pigment comprises a lipophilic or hydrophobic coating, the latter is preferably present in the fatty phase of the composition according to the invention.

According to a specific embodiment of the invention, the pigments can be coated according to the invention with at least one compound chosen from silicone surface agents; fluorinated surface agents; fluorosilicone surface agents; metal soaps; N-acylamino acids or salts thereof; lecithin and derivatives thereof; isopropyl titanium triisostearate; isostearyl sebacate; natural vegetable or animal waxes; polar synthetic waxes; fatty esters; phospholipids; and mixtures thereof.

### Silicone Surface Agent

According to a specific embodiment, the pigments can be completely or partially surface-treated with a compound of silicone nature.

The silicone surface agents can be chosen from organopolysiloxanes, silane derivatives, silicone-acrylate copolymers, silicone resins and mixtures thereof.

The term “organopolysiloxane compound” means a compound having a structure comprising alternating silicon atoms and oxygen atoms and comprising organic radicals bonded to the silicon atoms.

### Nonelastomeric Organopolysiloxane

Mention may in particular be made, as nonelastomeric organopolysiloxanes, of polydimethylsiloxanes, polymethylhydrosiloxanes and polyalkoxydimethylsiloxanes. The alkoxy group can be represented by the R—O-radical such that R represents methyl, ethyl, propyl, butyl or octyl, 2-phenylethyl, 2-phenylpropyl or 3,3,3-trifluoropropyl radicals, aryl radicals, such as phenyl, tolyl or xylyl, or substituted aryl radicals, such as phenylethyl.

One method which makes it possible to surface-treat pigments with a polymethylhydrosiloxane consists in dispersing the pigments in an organic solvent and in then adding the silicone compound. On heating the mixture, covalent bonds are created between the silicone compound and the surface of the pigment.

According to a preferred embodiment, the silicone surface agent can be a nonelastomeric organopolysiloxane, in particular chosen from polydimethylsiloxanes.

### Alkylsilanes and Alkoxysilanes

Silanes having alkoxy functionality are described in particular by Witucki in A Silane Primer, Chemistry and Applications of Alkoxysilanes, Journal of Coatings Technology, 65, 822, pages 57-60, 1993.

Alkoxysilanes, such as the alkyltriethoxysilanes and the alkyltrimethoxysilanes sold under the references Milquet A-137® (OSI Specialities) and Prosil 9202® (PCR), can be used for coating the pigments.

The use of alkylpolysiloxanes having a reactive end group, such as alkoxy, hydroxyl, halogen, amino or imino, is described in the application

JP H07-196946. They are also suitable for treating the pigments.

### Silicone-Acrylate Polymers

Use may be made of grafted silicone-acrylic polymers having a silicone backbone as described in the patents U.S. Pat. Nos. 5,725,882, 5,209,924, 4,972,037, 4,981,903, 4,981,902, 5,468,477, and in patents U.S. Pat. No. 5,219,560 and EP 0 388 582.

Other silicone-acrylate polymers can be silicone polymers comprising, in their structure, the unit of following formula (II):

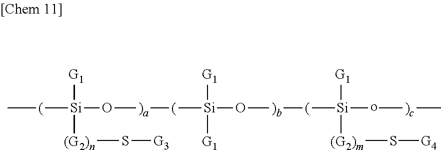

in which the G1 radicals, which are identical or different, represent hydrogen or a C₁-C₁₀ alkyl radical or else a phenyl radical; the G2 radicals, which are identical or different, represent a C₁-C₁₀ alkylene group; G3 represents a polymeric residue resulting from the (homo) polymerization of at least one ethylenically unsaturated anionic monomer; G4 represents a polymeric residue resulting from the (homo) polymerization of at least one ethylenically unsaturated hydrophobic monomer; m and n are equal to 0 or 1; a is an integer ranging from 0 to 50; b is an integer which can be between 10 and 350 and c is an integer ranging from 0 to 50, with the proviso that one of the parameters a and c is other than 0.

Preferably, the unit of formula (I) above has at least one, and even more preferentially all, of the following characteristics:
the G₁ radicals denote an alkyl radical, preferably the methyl radical;
n is nonzero, and the G₂ radicals represent a divalent C₁-C₃ radical, preferably a propylene radical;
G₃ represents a polymeric radical resulting from the (homo) polymerization of at least one monomer of the ethylenically unsaturated carboxylic acid type, preferably acrylic acid and/or methacrylic acid;
G₄ represents a polymeric radical resulting from the (homo) polymerization of at least one monomer of the (C₁-C₁₀)alkyl (meth)acrylate type, preferably of the isobutyl or methyl (meth)acrylate type.

Examples of silicone polymers corresponding to the formula (I) are in particular polydimethylsiloxanes (PDMSs) to which mixed polymer units of the poly(meth)acrylic acid type and of the polymethyl (meth)acrylate type are grafted via a thiopropylene-type connecting link.

Other examples of silicone polymers corresponding to the formula (I) are in particular polydimethylsiloxanes (PDMSs) to which polymer units of the polyisobutyl (meth)acrylate type are grafted via a thiopropylene-type connecting link.

### Silicone Resins

The silicone surface agent may be chosen from the silicone resins as defined previously.

### Fluorinated Surface Agent

The pigments can be completely or partially surface-treated with a compound of fluorinated nature.

The fluorinated surface agents can be chosen from perfluoroalkyl phosphates, perfluoropolyethers, polytetrafluoroethylenes (PTFEs), perfluoroalkanes, perfluoroalkyl silazanes, polyhexafluoropropylene oxides or polyorganosiloxanes comprising perfluoroalkyl perfluoropolyether groups.

The term “perfluoroalkyl radical” means an alkyl radical in which all the hydrogen atoms have been replaced with fluorine atoms.

Perfluoropolyethers are described in particular in the patent application EP 0 486 135 and are sold under the trade name Fomblin by Montefluos.

Perfluoroalkyl phosphates are described in particular in the application JP H05-86984. The perfluoroalkyl phosphate diethanolamines sold by Asahi Glass under the reference Asahi Guard AG530® can be used.

Mention may be made, among the linear perfluoroalkanes, of perfluorocycloalkanes, perfluoro(alkylcycloalkanes), perfluoropolycycloalkanes, perfluorinated aromatic hydrocarbons (perfluoroarenes) and organoperfluorinated hydrocarbon-based compounds comprising at least one heteroatom.

Mention may be made, among the perfluoroalkanes, of the series of the linear alkanes, such as perfluorooctane, perfluorononane or perfluorodecane.

Among the perfluorocycloalkanes and perfluoro (alkylcycloalkanes), mention may be made of perfluorodecalin sold under the name Flutec PP5 GMP by Rhodia, perfluoro(methyldecalin) and perfluoro (C₃-C₅ alkylcyclohexanes) such as perfluoro (butylcyclohexane).

Mention may be made, among the perfluoropolycycloalkanes, of bicyclo[3.3.1]nonane derivatives, such as perfluorotrimethylbicyclo[3.3.1]nonane, adamantane derivatives, such as perfluorodimethyladamantane, and perfluorinated derivatives of hydrogenated phenanthrene, such as tetracosafluorotetradecahydrophenanthrene.

Mention may be made, among the perfluoroarenes, of perfluorinated derivatives of naphthalene, such as perfluoronaphthalene and perfluoro-1-methylnaphthalene. Mention may be made, as examples of commercial references of pigments treated with a fluorinated compound, of:
yellow iron oxide/perfluoroalkyl phosphate, sold under the reference PF 5 Yellow 601® by Daito Kasei;
red iron oxide/perfluoroalkyl phosphate, sold under the reference PF 5 Red R 516L® by Daito Kasei;
black iron oxide/perfluoroalkyl phosphate sold under the reference PF 5 Black BL100® by Daito Kasei;
titanium dioxide/perfluoroalkyl phosphate sold under the reference PF 5 TiO2 CR 50® by Daito Kasei;
yellow iron oxide/perfluoropolymethyl isopropyl ether sold under the reference Iron Oxide Yellow BF-25-3® by Toshiki;
DC Red 7/perfluoropolymethyl isopropyl ether sold under the reference D&C Red 7 FHC® by Cardre Inc.; and
DC Red 6/PTFE sold under the reference T 9506® by Warner-Jenkinson.

### Fluorosilicone Surface Agent

The pigments can be completely or partially surface-treated with a compound of fluorosilicone nature.

The fluorosilicone compound can be chosen from perfluoroalkyl dimethicones, perfluoroalkylsilanes and perfluoroalkyltrialkoxysilanes.

Perfluoroalkyl silanes that may be mentioned include the products LP-IT® and LP-4TR sold by Shin-Etsu Silicone.

As examples of commercial references of pigments treated with a fluorosilicone compound, mention may be made of titanium dioxide/fluorosilicone sold under the reference Fluorosil Titanium Dioxide 100TAR by Advanced Dermaceuticals International Inc.

### Other Lipophilic Surface Agents

The hydrophobic treatment agent can also be chosen from:
(i) metal soaps, such as aluminium dimyristate and the aluminium salt of hydrogenated tallow glutamate.

Mention may in particular be made, as metal soaps, of metal soaps of fatty acids having from 12 to 22 carbon atoms and in particular those having from 12 to 18 carbon atoms.

The metal of the metal soap can in particular be zinc or magnesium.

Use may be made, as metal soap, of zinc laurate, magnesium stearate, magnesium myristate, zinc stearate and their mixtures.

The hydrophobic treatment agent can also be chosen from ii) fatty acids, such as lauric acid, myristic acid, stearic acid or palmitic acid.

The hydrophobic treatment agent can also be chosen from iii) N-acylated amino acids or their salts, which can comprise an acyl group having from 8 to 22 carbon atoms, such as, for example, a 2-ethylhexanoyl, caproyl, lauroyl, myristoyl, palmitoyl, stearoyl or cocoyl group.

The amino acid can, for example, be lysine, glutamic acid or alanine.

The salts of these compounds can be the aluminium, magnesium, calcium, zirconium, zinc, sodium or potassium salts.

Thus, according to a particularly preferred embodiment, an N-acylated amino acid derivative can in particular be a glutamic acid derivative and/or a salt thereof and more particularly a stearoyl glutamate, for example aluminium stearoyl glutamate.

The hydrophobic treatment agent can also be chosen from iv) lecithin and derivatives thereof.

The hydrophobic treatment agent can also be v) isopropyl titanium triisostearate.

As examples of isopropyl titanium triisostearate (ITT)-treated pigments, mention may be made of those sold under the commercial references BWBO-12® (Iron oxide CI77499 and isopropyl titanium triisostearate), BWYO-12® (Iron oxide CI77492 and isopropyl titanium triisostearate) and BWRO-12® (Iron oxide CI77491 and isopropyl titanium triisostearate) by Kobo.

The hydrophobic treatment agent can also be vi) isostearyl sebacate.

The hydrophobic treatment agent can also be chosen from vii) natural vegetable or animal waxes or polar synthetic waxes.

The hydrophobic treatment agent can also be chosen from viii) fatty esters, in particular jojoba esters.

The hydrophobic treatment agent can also be chosen from ix) phospholipids.

The waxes mentioned in the compounds cited above can be those generally used in the cosmetics field, as are defined subsequently.

They can in particular be hydrocarbon-based, silicone and/or fluorinated, optionally comprising ester or hydroxyl functional groups. They can also be of natural or synthetic origin.

The term “polar wax” means a wax containing chemical compounds comprising at least one polar group. Polar groups are well known to a person skilled in the art; they can, for example, be alcohol, ester or carboxylic acid groups. Polyethylene waxes, paraffin waxes, microcrystalline waxes, ozokerite or Fischer-Tropsch waxes are not included among polar waxes.

In particular, polar waxes have a mean Hansen solubility parameter δa at 25° C. such that δa>0 (J/cm³)^{1/2} and better still δa>1 (J/cm³)^{1/2}:

$${δ}_{a} = \sqrt{{δ}_{p}^{2} + {δ}_{h}^{2}}$$

where δp and δh are, respectively, the polar contributions and the contributions of types of specific interactions to the Hansen solubility parameters.

The definition of solvents in the three-dimensional solubility space according to Hansen is described in the paper by C. M. Hansen, The three-dimensional solubility parameters, J. Paint Technol., 39, 105 (1967):
δh characterizes the forces of specific interactions (of the hydrogen bond type, acid/base interaction or donor/acceptor bonds, etc.);
δp characterizes the Debye interaction forces between permanent dipoles and also the Keesom interaction forces between induced dipoles and permanent dipoles.

The parameters δp and δh are expressed in (J/cm³)^{1/2}.

A polar wax is formed in particular of molecules comprising, besides carbon and hydrogen atoms in their chemical structure, heteroatoms (such as O, N and P). Mention may in particular be made, as nonlimiting illustration of these polar waxes, of natural polar waxes, such as beeswax, lanolin wax, orange wax, lemon wax and Chinese insect waxes, rice bran wax, carnauba wax, candelilla wax, ouricury wax, cork fiber wax, sugar cane wax, Japan wax, sumac wax or montan wax.

According to a specific embodiment, the pigments can be coated with at least one compound chosen from silicone surface agents; fluorinated surface agents; N-acylated amino acids or their salts; isopropyl titanium triisostearate; natural vegetable or animal waxes; fatty esters; and mixtures thereof.

According to a particularly preferred embodiment, the pigments can be coated with an N-acylated amino acid and/or a salt thereof, in particular with a glutamic acid derivative and/or a salt thereof, or with a fatty ester, in particular with a jojoba ester.

According to a more particularly preferred embodiment, the pigments can be coated with an N-acylated amino acid and/or a salt thereof, in particular with a glutamic acid derivative and/or a salt thereof, especially a stearoyl glutamate, such as, for example, aluminium stearoyl glutamate.

Mention may more particularly be made, as examples of coated pigments according to the invention, of titanium dioxides and iron oxides coated with aluminium stearoyl glutamate, for example sold under the reference NAI by Miyoshi Kasei.

Pigments not Coated with a Hydrophobic Compound

As stated above, a composition can additionally contain pigments not coated with a lipophilic or hydrophobic compound.

These other pigments can be coated with a hydrophilic compound or be uncoated. These pigments can be mineral pigments, in particular as defined above.

These pigments can also be organic pigments.

The term “organic pigment” means any pigment that satisfies the definition in Ullmann's encyclopaedia in the chapter on organic pigments. The organic pigment can in particular be chosen from nitroso, nitro, azo, xanthene, quinoline, anthraquinone, phthalocyanine, of metal complex type, isoindolinone, isoindoline, quinacridone, perinone, perylene, diketopyrrolopyrrole, thioindigo, dioxazine, triphenylmethane or quinophthalone compounds.

The organic pigment(s) can be chosen, for example, from carmine, carbon black, aniline black, melanin, azo yellow, quinacridone, phthalocyanine blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenol derivatives as are described in the patent FR 2 679 771.

These pigments can also be in the form of composite pigments as are described in the patent EP1184426. These composite pigments can be composed in particular of particles comprising an inorganic core at least partially covered with an organic pigment and at least one binder providing the fixing of the organic pigments to the core.

The pigment can also be a lake. The term “lake” is understood to mean insolubilized dyes adsorbed on insoluble particles, the assembly thus obtained remaining insoluble during use.

The inorganic substrates onto which the dyes are adsorbed are, for example, alumina, silica, calcium sodium borosilicate or calcium aluminium borosilicate, and aluminium. Mention may be made, among the organic dyes, of cochineal carmine. Mention may also be made of the products known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 1 O (CI 77 002), D&C Green 3 (CI 42 053), D&C Blue 1 (CI 42 090).

An example of a lake that may be mentioned is the product known under the name D&C Red 7 (CI 15 850:1).

### Nature of the Hydrophilic Coating

As stated above, these other pigments can be coated with a hydrophilic compound. Said hydrophilic compound, which makes it possible to surface treat a pigment in order to optimize its dispersion in the gelled aqueous phase, is more particularly chosen from biological polymers, carbohydrates, polysaccharides, polyacrylates or polyethylene glycol derivatives.

Mention may be made, as examples of biological polymers, of polymers based on monomers of carbohydrate type.

Mention may more particularly be made of biosaccharide gum; chitosans and derivatives thereof, such as butoxy chitosan, carboxymethyl chitosan, carboxybutyl chitosan, chitosan gluconate, chitosan adipate, chitosan glycolate, chitosan lactate, and the like; chitins and derivatives thereof, such as carboxymethyl chitin or chitin glycolate; cellulose and derivatives thereof, such as cellulose acetate; microcrystalline cellulose; distarch phosphate; sodium hyaluronate; soluble proteoglycans; galactoarabinans; glycosaminoglycans; glycogen; sclerotium gum; dextran; starch and derivatives thereof; and mixtures thereof.

Mention may in particular be made, as examples of carbohydrates, of polyhydroxyaldehydes or polyhydroxyketones, of general formula: Cx (H2O) y in which x and y can range from 1 to 1 000 000.

The carbohydrates can be monosaccharides, disaccharides or polysaccharides. Mention may in particular be made, as examples of carbohydrates, of amylodextrins, beta-glucans, cyclodextrins, modified corn starch, glycogen, hyaluronic acid, hydroxypropylcyclodextrin, lactose, maltitol, guanosine, glyceryl starch, Triticum vulgare starch, trehalose, sucrose and derivatives thereof, raffinose or sodium chondroitin sulfate.

C₁-C₂₀ Alkylene glycols or C₁-C₂₀ alkylene glycol ethers, alone or in combination with tri(C₁-C₂₀)alkylsilanes, may also be used as surface treatment agents.

Mention may be made, as examples, of the pigments surface-treated with PEG alkyl ether alkoxysilane, for example the pigments treated with PEG-8 methyl ether triethoxysilane which are sold by Kobo under the name SW pigments.

Silicones, such as dimethicones having hydrophilic groups, also known under the name dimethicone copolyols or alkyl dimethicone copolyols, may also be suitable for the invention as surface treatment agents. In particular, such dimethicones may comprise, as repeating units, C₁-C₂₀ alkylene oxides, such as ethylene or propylene oxides.

An example that may be mentioned is the pigment treated with PEG-12-dimethicone, sold by Sensient Corporation under the name LCW AQ® Pigment.

The amount of pigments coated with at least one hydrophilic compound and/or of uncoated pigments is particularly dictated by the intended use of the cosmetic composition in question, and the adjustment of this amount obviously falls within the competence of the formulator of the composition.

According to a particular embodiment, the composition additionally comprises at least one pigment chosen from titanium dioxides and/or iron oxides, in particular coated with a hydrophobic surface treatment agent, particularly with an N-acylated amino acid and/or a salt thereof, in particular with a glutamic acid derivative and/or a salt thereof, particularly a stearoyl glutamate, for example aluminium stearoyl glutamate.

### Additives

The compositions according to the invention can additionally comprise additives commonly used in care and/or makeup products, such as organic UV screening agents other than those described above; inorganic UV screening agents; moisturizing agents, such as polyols, for example glycerol, propanediol or pentylene glycol; fillers; dyestuffs; thickening or gelling agents; preservatives; chelating agents; fragrances; and mixtures thereof.

### Fillers

The compositions in accordance with the invention can also comprise at least one filler, of organic or inorganic nature, which makes it possible in particular to confer on them additional properties of improved stability, persistence, coverage and/or mattness. The term “filler” should be understood as meaning colourless or white solid particles of any shape which are provided in an insoluble form and dispersed in the medium of the composition. These particles, of mineral or organic nature, make it possible to confer body or firmness on the composition and/or softness and uniformity on the makeup.

The fillers used in the compositions according to the present invention can be of lamellar, globular, spherical or fibrous forms or of any other form intermediate between these defined forms.

The fillers according to the invention may or may not be surface-coated, and in particular they may be surface-treated with silicones, amino acids, fluorinated derivatives or any other substance which promotes the dispersion and the compatibility of the filler in the composition.

Mention may be made, as examples of inorganic fillers, of talc, mica, silica, hollow silica microspheres, kaolin, calcium carbonate, magnesium carbonate, hydroxyapatite, boron nitride, glass or ceramic microcapsules, composites of silica and of titanium dioxide, such as the TSG® series sold by Nippon Sheet Glass, or hydrophobic silica aerogels. Mention may be made, as examples of organic fillers, of powders formed of polyamide (Nylon® Orgasol from Atochem), of polyethylene, of polymethyl methacrylate, of polytetrafluoroethylene (Teflon®) or of acrylic acid copolymers (Polytrap® from Dow Corning), lauroyl lysine, hollow polymeric microspheres, such as those of polyvinylidene chloride/acrylonitrile, for example Expancel® (Nobel Industrie), Hexamethylene Diisocyanate/Trimethylol Hexyllactone copolymer powder (Plastic Powder® from Toshiki), silicone resin microbeads (Tospearl® from Toshiba, for example), synthetic or natural micronized waxes, metal soaps derived from organic carboxylic acids having from 8 to 22 carbon atoms, preferably from 12 to 18 carbon atoms, for example zinc stearate, magnesium stearate, lithium stearate, zinc laurate or magnesium myristate, Polypore® L 200 (Chemdal Corporation), or powders of crosslinked elastomeric organopolysiloxane coated with silicone resin, in particular with silsesquioxane resin, as described, for example, in the patent U.S. Pat. No. 5,538,793. It can also be a cellulose powder, such as that sold by Daito in the Cellulobeads range.

### Silica Particles

According to a preferred form, the composition according to the invention additionally comprises silica particles chosen from hydrophobic silica aerogel particles, silica particles other than the preceding ones, and mixtures thereof.

### i) Hydrophobic Silica Aerogels

Hydrophobic silica aerogels are porous materials obtained by replacing (in particular by drying) the liquid component of a silica gel with air. They are generally synthesized by a sol-gel process in a liquid medium and then dried, usually by extraction with a supercritical fluid, the one most commonly used being supercritical CO₂. This type of drying makes it possible to avoid shrinkage of the pores and of the material. The sol-gel process and the various drying operations are described in detail in Brinker C. J. and Scherer G. W., Sol-Gel Science, New York, Academic Press, 1990.

The hydrophobic silica aerogels used according to the present invention are preferably silylated silica aerogels (INCI name: Silica Silylate).

The term “hydrophobic silica” means any silica, the surface of which is treated with silylating agents, for example with halogenated silanes, such as alkylchlorosilanes, siloxanes, in particular dimethylsiloxanes, such as hexamethyldisiloxane, or silazanes, so as to functionalize the OH groups with Si—Rn silyl groups, for example trimethylsilyl groups.

As regards the preparation of hydrophobic silica aerogel particles which have been surface-modified by silylation, reference may be made to the document U.S. Pat. No. 7,470,725. Use will be made in particular of aerogel particles formed of hydrophobic silica which is surface-modified with trimethylsilyl groups (trimethylsiloxylated silica).

The term “hydrophobic aerogel particles” means any particle of the aerogel type exhibiting a water absorption capacity at the wet point of less than 0.1 ml/g, i.e. less than 10 g of water per 100 g of particle.

The absorption capacity, measured at the wet point and denoted WP, corresponds to the amount of a solvent (expressed in grams or in millilitres) which it is necessary to add to 1 g of particles in order to obtain a homogeneous paste. It is measured according to the “wet point” method or the method for determining the uptake of solvent (water or oil) of a powder described in the standard NF T 30-022. It corresponds to the amount of solvent adsorbed onto the available surface of the powder and/or absorbed by the powder by measurement of the wet point, described below:

A glass plate (25×25 mm) is deposited on a balance, an amount w of 1 g of powder is weighed out onto the glass plate and then a solvent (water or isononyl isononanoate, for example) is added dropwise. The solvent is added gradually to the powder, everything being regularly kneaded (every 3 to 4 drops) using a spatula. The addition of solvent is halted when a homogeneous paste is obtained. This paste must be able to be spread over the glass plate without cracks or the formation of lumps. The weight of solvent necessary to obtain the wet point is recorded. The mean over three tests will be taken. As the density of the solvent is known, the volume Vs (expressed in ml) of solvent used is deduced therefrom. The solvent uptake corresponds to the ratio Vs/w.

Preferably, the hydrophobic silica aerogel particles according to the invention preferably have an oil absorption capacity, measured at the wet point, ranging from 5 to 18 ml/g, preferably from 6 to 15 ml/g and better still from 8 to 12 ml/g.

The hydrophobic silica aerogel particles used in the present invention preferably have a specific surface area per unit of weight (SM) ranging from 200 to 1500 m²/g, preferably from 600 to 1200 m²/g and better still from 600 to 800 m²/g, and a size, expressed as the volume-mean diameter (D[0.5]), of less than 1500 μm and preferably ranging from 1 to 30 μm, preferably from 5 to 25 μm, better still from 5 to 20 μm and even better still from 5 to 15 μm.

The specific surface area per unit of weight can be determined by the nitrogen absorption method, known as the BET (Brunauer-Emmett-Teller) method, described in The Journal of the American Chemical Society, Vol. 60, page 309, February 1938 and corresponding to the international standard ISO 5794/1 (appendix D). The BET specific surface area corresponds to the total specific surface area of the particles under consideration. The sizes of the aerogel particles according to the invention can be measured by static light scattering using a commercial particle size analyser of Mastersizer 2000® type from Malvern. The data are processed on the basis of the Mie scattering theory. This theory, which is exact for isotropic particles, makes it possible to determine, in the case of non-spherical particles, an effective particle diameter. This theory is described in particular in the publication by Van de Hulst, H. C., Light Scattering by Small Particles, Chapters 9 and 10, Wiley, New York, 1957.

The hydrophobic silica aerogel particles used in the present invention may advantageously have a tapped density ranging from 0.02 g/cm³ to 0.10 g/cm³ and preferably from 0.02 g/cm³ to 0.08 g/cm³.

In the context of the present invention, this density may be assessed according to the following protocol, known as the tapped density protocol:

40 g of powder are poured into a measuring cylinder; the measuring cylinder is then placed on a Stav 2003® machine from Stampf Volumeter; the measuring cylinder is then subjected to a series of 2500 tapping actions (this operation is repeated until the difference in volume between two consecutive tests is less than 2%); the final volume Vf of tapped powder is then measured directly on the measuring cylinder. The tapped density is determined by the ratio m/Vf, in this case 40/Vf (Vf being expressed in cm³ and m in g).

According to one embodiment, the hydrophobic aerogel particles used in the present invention have a specific surface area per unit volume (S_{V}) ranging from 5 to 60 m²/cm³, preferably from 10 to 50 m²/cm³ and better still from 15 to 40 m²/cm³.

The specific surface area per unit volume is given by the relationship: S_{V}=S_{M}·ρ where ρ is the tapped density, expressed in g/cm³ and SM is the specific surface area per unit of weight, expressed in m²/g, as defined above.

According to a particular embodiment, the aerogel particles used are inorganic and are more particularly hydrophobic silica aerogel particles having the properties stated previously.

Mention may be made, as hydrophobic silica aerogels which can be used in the invention, for example, of the aerogel sold under the name VM-2260 (INCI name: Silica Silylate) by Dow Corning, the particles of which have an average size of approximately 1000 microns and a specific surface area per unit of weight ranging from 600 to 800 m²/g.

Mention may also be made of the aerogels sold by Cabot under the references Aerogel TLD 201®, Aerogel OGD 201®, Aerogel TLD 203®, Enova® Aerogel MT 1100 and Enova Aerogel MT 1200®.

Use will be made more particularly of the aerogel sold under the name VM-2270® (INCI name Silica Silylate), by Dow Corning, the particles of which have an average size ranging from 5 to 15 microns and a specific surface area per unit of weight ranging from 600 to 800 m²/g.

Use will also be made of the aerogel sold under the name Enova® Aerogel MT 1100® (INCI name: Silica Silylate), by Cabot, the particles of which have a mean size ranging from 2-25 microns and a specific surface area per unit of weight ranging from 600 to 800 m²/g.

The hydrophobic aerogel particles represent from 0.05% to 10% by weight, preferably from 0.1% to 8% by weight, better still from 0.2% to 5% by weight and more preferably from 0.3% to 3% by weight relative to the total weight of the composition.

### Other Silica Particles

The other silicas which can be used can be natural and untreated. Mention may thus be made of the silicas provided under the names Sillitin N85®, Sillitin N87®, Sillitin N82®, Sillitin V85® and Sillitin V88® by Hoffmann Mineral.

They can be fumed silicas.

The fumed silicas may be obtained by high-temperature hydrolysis of a volatile silicon compound in an oxyhydrogen flame, producing a finely divided silica. This process makes it possible in particular to obtain hydrophilic silicas which have a large number of silanol groups at their surface. It is possible to chemically modify the surface of said silica by chemical reaction generating a reduction in the number of silanol groups. It is possible in particular to substitute silanol groups with hydrophobic groups: a hydrophobic silica is then obtained.

The hydrophobic groups can be:
(a) trimethylsiloxy groups, which are in particular obtained by treatment of fumed silica in the presence of hexamethyldisilazane. Silicas thus treated are known as Silica Silylate according to the CTFA (6th Edition, 1995);
(b) dimethylsilyloxy or polydimethylsiloxane groups, which are in particular obtained by treatment of fumed silica in the presence of polydimethylsiloxane or dimethyldichlorosilane. Silicas thus treated are known as Silica Dimethyl Silylate according to the CTFA (6th Edition, 1995).

Mention may more particularly be made, as silica powders other than silicon aerogels, of:
the porous silica microspheres sold under the name Silica Beads SB-700® by Miyoshi; Sunsphere® H51 or Sunsphere® H33 by Asahi Glass;
the polydimethylsiloxane-coated amorphous silica microspheres sold under the names SA Sunsphere® H 33® and SA Sunsphere® H53@ by AGC SITECH;
precipitated silica microspheres, for example coated with mineral wax, such as polyethylene, and sold in particular under the name Acematt® OK 412® by Evonik Degussa.

Use will more particularly be made, as silica powder, of porous silica microspheres, such as those sold under the names Silica Beads SB-700® by Miyoshi and Sunsphere® H51 and Sunsphere® H33 by AGC SITECH.

The silica particles other than the hydrophobic silica aerogel particles are present in the composition according to the invention in a content ranging from 0.01% to 15% by weight, preferably ranging from 0.1% to 10% by weight and very preferentially ranging from 0.5% to 5% by weight, relative to the total weight of the composition.

According to a preferential form, the composition according to the invention will comprise a mixture comprising at least hydrophobic silica aerogel particles, such as those described above, and other silica particles, such as those described above, in particular porous silica microspheres.

### Additional Dyestuffs

A composition according to the invention may also comprise at least one additional dyestuff, preferably in a proportion of at least 0.01% by weight relative to the total weight of the composition.

For obvious reasons, this amount is liable to vary significantly with regard to the desired intensity of the colour effect and to the colour intensity provided by the dyestuffs under consideration, and its adjustment clearly falls within the competence of a person skilled in the art.

The additional dyestuffs that are suitable for use in the invention may be water-soluble, but may also be liposoluble.

For the purposes of the invention, the term “water-soluble dyestuff” means any natural or synthetic, generally organic compound, which is soluble in an aqueous phase or water-miscible solvents and which is capable of imparting colour.

Mention may in particular be made, as water-soluble dyes suitable for the invention, of synthetic or natural water-soluble dyes, such as, for example, FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanin (beetroot), carmine, copper chlorophyllin, methylene blue, anthocyanins (enocyanin, black carrot, hibiscus or elder), caramel or riboflavin.

The water-soluble dyes are, for example, beetroot juice and caramel.

For the purposes of the invention, the term “liposoluble dyestuff” means any natural or synthetic, generally organic compound, which is soluble in an oily phase or in solvents that are miscible with a fatty substance, and which is capable of imparting colour.

As liposoluble dyes that are suitable for use in the invention, mention may in particular be made of synthetic or natural liposoluble dyes, for instance DC Red 17, DC Red 21, DC Red 27, DC Green 6, DC Yellow 11, DC Violet 2, DC Orange 5, Sudan red, carotenes (β-carotene, lycopene), xanthophylls (capsanthin, capsorubin, lutein), palm oil, Sudan brown, quinoline yellow, annatto and curcumin.

### Form of the Composition

The composition of the invention can be in the form of an anhydrous composition, a water-in-oil emulsion or an oil-in-water emulsion.

According to a first embodiment of the invention, the composition is a single-phase oily composition.

According to another advantageous embodiment of the invention, the composition comprises an aqueous phase, in which case it is preferably in the form of a water-in-oil emulsion, or possibly a composition having several separate phases (such as a two-phase composition).

The term “water-in-oil emulsion” or W/O emulsion means a composition comprising an oily phase and an aqueous phase which are immiscible; the aqueous phase being dispersed in the form of droplets in the oily phase (described as continuous) so as to obtain a macroscopically homogeneous composition.

The presentation forms having a continuous oily phase are favoured in the case of the composition of the invention, where the persistence performance driver is provided by the natural resin solubilized in the oily phase. These forms additionally promote the dispersion of the pigments, the homogeneity thereof and thus optimize the coverage obtained for the film obtained after applying the composition according to the invention (as demonstrated in the examples).

The composition of the invention is particularly suitable for producing cosmetic products with fluid textures. Advantageously, the composition according to the invention is more particularly in the form of a viscoelastic to viscous liquid, the G* modulus (viscoelastic modulus) of which is between 0.1 and 20 000 Pa, more particularly between 1 and 5000 Pa, or even between 10 and 1000 Pa. The G* modulus is measured with a controlled stress rheometer and the values are taken on the viscoelastic plateau at 25° C.

### Applications

According to one embodiment, a composition of the invention can advantageously be provided in the form of a composition for caring for the skin of the body or of the face, in particular of the face.

According to another embodiment, a composition of the invention can advantageously be provided in the form of a composition for making up keratin materials, in particular the skin of the body or of the face, in particular of the face.

Thus, according to a sub-mode of this embodiment, a composition of the invention may advantageously be in the form of a makeup base composition.

A composition of the invention may advantageously be in the form of a liquid product for making up the lips, in particular in the form of a liquid lipstick.

According to another sub-mode of this embodiment, a composition of the invention may advantageously be in the form of a composition for making up the skin and especially the face. It can thus be a foundation, an eyeshadow or a blusher.

It may also be a mascara, an eyeliner, a concealer or corrector, an eyebrow product, a skincare product, a sun protection product, a hygiene product, or else a hair shaping product, or a hair dyeing product; or even a nail varnish.

Such compositions are particularly prepared according to the general knowledge of a person skilled in the art.

The invention is illustrated in greater detail by the examples presented below. Unless otherwise indicated, the amounts indicated are expressed as mass percentages. The examples that follow allow the invention to be understood more clearly, without, however, being limiting in nature.

### EXAMPLES

### Ingredients:

TABLE 1

| | | PROTIUM | | |
|---|---|---|---|---|
| | | HEPTA- | | |
| | GLYCERYL | PHYLLUM | | |
| | ROSINATE | RESIN | CANDELILLA | |
| | Resin | Resin | Resin | |
| | C | B | A | Wax |
| Melting | — | — | — | 66-71 |
| temperature (Mp) °C | | | | |
| Softening | 86 | 42 | 45-52 | |
| point °C | 84-88 | 40-45 | | |
| Glass transition | 43 | 51 | 34 | — |
| temperature | 40-45 | 48-55 | | |
| (Tg) °C | | | | |

TABLE 2

| | INCI name | Trade name | Supplier |
|---|---|---|---|
| Resin | | | |
| A | EUPHORBIA CERIFERA | CANDELILLA | JAPAN NATURAL |
| | (CANDELILLA) WAX | RESIN E-1 | PRODUCTS |
| | EXTRACT | | |
| B | PROTIUM | CITROBREU | CITROLEO |
| | HEPTAPHYLLUM RESIN | | |
| C | GLYCERYL ROSINATE | RESIESTER | LURESA RESINAS |
| | | GUM A 35 | S.L. |
| Gelling | | | |
| agent | | | |
| D | ETHYLCELLULOSE | AQUALON EC N7 | ASHLAND |
| | | PHARM | |
| E | MYRISTOYL PULLULAN | MYRISTOYL | KATAKURA |
| | | PULLULAN | CHIKKARIN |
| F | C1-5 | N HANCE AG 200 | ASHLAND |
| | ALKYLGALACTOMANNAN | (LOW MOLECULAR | |
| | | WEIGHT) | |

### Preparation of the Compositions

Procedure and equipment used for each test:

Each resin (A, B, C) and each gelling agent (D, E, F, G, H) were used in mixtures of ethanol and isododecane at ambient temperature, in proportions by weight indicated in the following tables, and according to the protocol described below:
Mix the resin with the solvents under magnetic stirring at 1200 rpm for 24 h at ambient temperature of 25° C.
Add the gelling agent by sprinkling into the resin/solvents mixture.
Leave stirring until the resin is completely solubilized and the polymer is deployed.
Weigh the assembly (beaker+mixture), readjust the amount of volatile solvents (if a slight loss of volatiles is observed).
The following coloured starting materials are then added: the RED 7 organic pigment or the coated iron oxide mineral pigments using a rotor-stator at ambient temperature (25° C.).

### Protocol for Evaluating the Dry Friction Resistance and the Friction Resistance in the Presence of Oil

Some compositions are assessed for their resistance to friction by colorimetric measurements on dry film before and after abrasion, for which test the protocol is described in detail below.

### Protocol for Spreading the Compositions as a Film:

The products are spread on a spreading bench (Elcometer 4340 Applicator), making it possible to regulate the speed of spreading and also the distance of spreading. The bench is equipped with a suction system connected to a pump so that the support on which the product is being spread does not move. Uncoated contrast cards with a black background and white background are used (1 byko-chart, uncoated N2A, code 2831). The thickness of spreading can be regulated using the square spreader placed on the support so as to spread by levelling when the platform is set in motion. Each edge face of the spreader enables spreading with a different thickness ranging from 25 μm to 200 μm. The thickness chosen is 25 μm so as to be close to a film thickness in vivo. A weight of 960 g is added over the spreader during the spreading. The spreading rate is set at 1″/sec, i.e. 2.54 cm/s. The films are dried for 24 h at 34° C. and ambient RH (50% RH) on a heating plate.

### Test Protocol for Resistance to Friction:

The friction resistance test is performed by colorimetric measurements on dry film before and after abrasion. The abrasion is carried out by attaching a strip of tissue handkerchief (Chicopee® Veraclean™ Polish Plus) to the 25 μm spreader edge face. A weight of 960 g is added over the spreader during the abrasion. The bench speed is set at 2.54 cm/s. The colour is measured before and after abrasion using a Konica Minolta CM-700d spectrophotometer. Measurement with contact makes it possible to guarantee the absence of parasitic light.

Settings chosen: Aperture 8 mm; uncertainty: 0.04; SCI/SCE measurement; d/8° geometry.

The measurements of the colours on the two backgrounds (black background BB and white background WB) make it possible to characterize the coverage of a foundation by calculating the contrast ratio (CR %), i.e. YBB/YWB×100, where YBB and YWB are the luminance values measured on the black background and on the white background, respectively, these values being higher with greater foundation coverage.

In order to asses the friction resistance, the contrast ratio before friction (CR dry deposit, %) and after abrasion (CR fric dry deposit, %), respectively, are measured. The ratio [CR fric dry deposit/CR dry deposit]*100, as a percentage, indicates the resistance of the film to friction: the higher this ratio, the more resistant the film is to friction.

Note: Regarding the acquisition of each contrast ratio value, at least 2 contrast cards are used for each composition and are assessed with 3 CR measurements on each card. Each CR value therefore represents a mean of 6 measurements.

The friction resistance of the dry film was tested for two types of coloured compositions 1) with organic pigments and then 2) with iron oxides (foundation prototypes).

The resistance of the film to olive oil was tested for compositions coloured with organic pigments (lip product prototypes).

### In Vitro Test of Fragmentation on FP40 Elastomeric Substrate-Evaluation of the Adhesion and Cohesion Properties of Deposits

Using an aluminium stencil (orifice 20 mm long and 10 mm wide, thickness of 100 μm), the film is cast manually with a pipette onto an FP40 support (test specimen 60 mm long and 10 mm wide, the FP40 substrate is made of butadiene-nitrile elastomer and contains a plasticizer (diethylhexyl sebacate); supplied by BRAMMER, Reference product JOINT DIVERS PLAQUE (MISCELLANEOUS PLATE SEAL) HPYF000504N4101, 60X1000x2 strip made of FP40, brand BUSAK-LU), then levelled off in order to obtain a film with a thickness of 100 μm. The test specimens are then left to dry for 24 h at 35° C. on a hot plate. The fragmentation test is carried out using a TA.XT texturometer from Stable Micro Systems with tensile modules. The test specimen is placed between the jaws at 1 cm from each side of the deposit during the tensile test, it is then stressed to a deformation of 100% at a constant speed of 300 mm/min. The resistance to fragmentation is observed by eye, and graded between 1 (whole of the deposit is fragmented) and 5 (complete absence of cracks). Examples of visual effects associated with the various scores are presented below.

TABLE 3

| Score | 1 | 2 | 3 | 4 | 5 |
|---|---|---|---|---|---|
| Description | No | Poor | Medium | Good | Excellent |
| | resistance, | resistance | resistance | resistance | resistance |
| | adhesion or | (detachments | (presence | (few small | (neither |
| | cohesion | or numerous | of several | local cracks | detachment |
| | (detachments | cracks over | cracks over | over less | nor cracks) |
| | and cracks) | the entire | at least | than 50% | |
| | | surface) | 50% of the | of the | |
| | | | surface) | surface) | |

### Examples 1 to 23—Results of Friction Resistance of the Compositions Comprising Candelilla Resin and/or Ethylcellulose

TABLE 4

| | Candelilla | Ethylcellulose | | | RED 7 | [CR Fric | [CR Fric | |
|---|---|---|---|---|---|---|---|---|
| | resin (A), | N7 (D) | Ethanol | Isododecane | CI 15850 | Dry/CR | Olive Oil/CR | Fragmentation |
| Test | (wt %) | (wt %) | (wt %) | (wt %) | (wt %) | Dry] (%) | Dry] (%) | resistance |
| 1 | 0 | 0 | 45 | 45 | 10 | 50.0 ± 2.1 | 13.8 ± 2.5 | — |
| (OI) | | | | | | | | |
| 2 | 5 | 0 | 42.5 | 42.5 | 10 | 61.2 ± 3.9 | 24.9 ± 3.9 | 3 |
| (OI) | | | | | | | | |
| 3 | 10 | 0 | 40 | 40 | 10 | 80.3 ± 3.6 | 28.8 ± 3.8 | 4 |
| (OI) | | | | | | | | |
| 4 | 15 | 0 | 37.5 | 37.5 | 10 | 94.0 ± 5.2 | 46.8 ± 0.7 | 5 |
| (OI) | | | | | | | | |
| 5 | 0 | 5 | 42.5 | 42.5 | 10 | 99.1 ± 2.2 | 40.5 ± 3.9 | 1 |
| (OI) | | | | | | | | |
| 6 | 0 | 10 | 40 | 40 | 10 | 100.3 ± 1.7 | 88.5 ± 2.8 | 1 |
| (OI) | | | | | | | | |
| 7 | 0 | 15 | 37.5 | 37.5 | 10 | 98.8 ± 2.8 | 99.7 ± 1.0 | 1 |
| (OI) | | | | | | | | |
| 8 | 0.5 | 2 | 44.25 | 44.25 | 10 | 72.73 ± 2.6 | 36.17 ± 3.2 | 2 |
| (Ex) | | | | | | | | |
| 9 | 1 | 5 | 42 | 42 | 10 | 97.82 ± 2.3 | 56.92 ± 2.6 | 2 |
| (Ex) | | | | | | | | |
| 10 | 5 | 5 | 40 | 40 | 10 | 99.2 ± 0.8 | 43.4 ± 6.5 | 3 |
| (Ex) | | | | | | | | |
| 11 | 10 | 1 | 39.5 | 39.5 | 10 | 100.9 ± 2.0 | — | — |
| (Ex) | | | | | | | | |
| 12 | 10 | 2.5 | 38.75 | 38.75 | 10 | 98.7 ± 0.6 | — | — |
| (Ex) | | | | | | | | |
| 13 | 10 | 5 | 37.5 | 37.5 | 10 | 98.8 ± 1.1 | 70.9 ± 3.3 | 4 |
| (Ex) | | | | | | | | |
| 14 | 10 | 10 | 35 | 35 | 10 | 99.9 ± 1.7 | — | — |
| (Ex) | | | | | | | | |
| 15 | 10 | 15 | 32.5 | 32.5 | 10 | 100.1 ± 0.8 | — | — |
| (Ex) | | | | | | | | |
| 16 | 10 | 20 | 30 | 30 | 10 | 99.8 ± 1.2 | — | — |
| (Ex) | | | | | | | | |

The compositions outside the invention containing only candelilla resin exhibit a lack of resistance in the presence of olive oil. The compositions outside the invention containing only ethylcellulose exhibit a lack of fragmentation resistance, with the appearance of a very high degree of cracking of the deposit which is synonymous with a lack of comfort on the skin.

Only the compositions of the invention, containing combinations of candelilla resin and ethylcellulose simultaneously exhibit excellent dry friction resistance, good resistance in the presence of olive oil and a good fragmentation resistance.

TABLE 5

| | Gelling agent | | Candelilla | | | RED 7 | [CR Fric | [CR Fric Olive |
|---|---|---|---|---|---|---|---|---|
| | (Type and | | resin, | Ethanol | Isododecane | CI 15850 | Dry/CR | Oil/CR Dry] |
| Tests | wt %) | | (wt %) | (wt %) | (wt %) | (wt %) | Dry] | (%) |
| 1 (OI) | — | 0 | 0 | 45 | 45 | 10 | 50.0 ± 2.1 | 13.8 ± 2.5 |
| 3 (OI) | — | 0 | 10 | 40 | 40 | 10 | 80.3 ± 3.6 | 28.8 ± 3.8 |
| 4 (OI) | — | 0 | 15 | 37.5 | 37.5 | 10 | 94.0 ± 5.2 | 46.8 ± 0.7 |
| 17 (OI) | Myristoyl | 5 | 0 | 42.5 | 42.5 | 10 | 53.2 ± 8.3 | 23.5 ± 2.3 |
| | pullulan | | | | | | | |
| 18 (Ex) | Myristoyl | 5 | 10 | 37.5 | 37.5 | 10 | 100.7 ± 2.7 | 75.9 ± 4.8 |
| | pullulan | | | | | | | |
| 19 (OI) | C1-5 Alkyl | 5 | 0 | 42.5 | 42.5 | 10 | 95.3 ± 4.1 | 43.2 ± 0.5 |
| | galactomannan | | | | | | | |
| 20 (Ex) | C1-5 Alkyl | 5 | 10 | 37.5 | 37.5 | 10 | 100.9 ± 2.7 | 80.7 ± 6.4 |
| | galactomannan | | | | | | | |

The formulae of the invention, containing candellila resin combined with myristoyl pullulan or with C1-5 alkyl galactomannan also exhibit good dry resistance and good resistance in the presence of olive oil.

TABLE 6

| | Candelilla | Ethylcellulose | | | Iron | [CR Fric | |
|---|---|---|---|---|---|---|---|
| | resin (A), | N7 (D) | Ethanol | Isododecane | oxides * | Dry/CR | |
| Test | (wt %) | (wt %) | (wt %) | (wt %) | (wt %) | Dry] (%) | SD |
| 21 (Ex) | 10 | 2.5 | 38.75 | 38.75 | 10 | 99.8 | 6.3 |
| 22 (Ex) | 10 | 5 | 37.5 | 37.5 | 10 | 98.5 | 4.8 |
| 23 (Ex) | 10 | 15 | 32.5 | 32.5 | 10 | 100.6 | 8.0 |

* Coated iron oxides having the INCI name: IRON OXIDES (and) DISODIUM STEAROYL GLUTAMATE (and) ALUMINUM HYDROXIDE.

The compositions of the invention containing iron oxides in the presence of resin and modified polysaccharide also show excellent dry friction resistance (around 100%).

### Examples 24 to 26—Results of Dry Friction Resistance or Friction Resistance with Olive Oil in the Presence of Glyceryl Rosinate

TABLE 7

| | | | | | | RED 7 | [CR Fric | [CR Fric |
|---|---|---|---|---|---|---|---|---|
| | | | Glyceryl | | | CI | Dry/CR | Olive Oil/ |
| | Gelling agent | | rosinate | Ethanol | Isododecane | 15850 | Dry] | CR Dry] |
| Test | (Type and wt %) | | (wt %) | (wt %) | (wt %) | (wt %) | (%) | (%) |
| 24 | Ethyl | 5 | 10 | 37.5 | 37.5 | 10 | 99.8 ± | 52.3 ± |
| (Ex) | cellulose | | | | | | 1.0 | 2.2 |
| 25 | Myristoyl | 5 | 10 | 37.5 | 37.5 | 10 | 101.8 ± | — |
| (Ex) | pullulan | | | | | | 1.9 | |
| 26 | C1-5 Alkyl | 5 | 10 | 37.5 | 37.5 | 10 | 99.7 ± | — |
| (Ex) | galactomannan | | | | | | 1.6 | |

The compositions of the invention containing a modified polysaccharide combined with glyceryl rosinate resin exhibit excellent dry friction resistance and good resistance in the presence of oil.

### Examples 27 to 29 Results of Dry Friction Resistance or Friction Resistance with Olive Oil in the Presence of Protium Heptaphyllum

TABLE 8

| | | | | | | | | [CR Fric |
|---|---|---|---|---|---|---|---|---|
| | | | Protium | | | RED 7 | [CR Fric | Olive Oil/ |
| | Gelling agent | | heptaphyllum | Ethanol | Isododecane | CI 15850 | Dry/CR Dry] | CR Dry] |
| Test | (Type and wt %) | | (wt %) | (wt %) | (wt %) | (wt %) | (%) | (%) |
| 27 (Ex) | Ethyl | 5 | 10 | 37.5 | 37.5 | 10 | 99.3 ± 1.3 | 65.4 ± 2.3 |
| | cellulose | | | | | | | |
| 28 (Ex) | Myristoyl | 5 | 10 | 37.5 | 37.5 | 10 | 99.7 ± 1.8 | — |
| | pullulan | | | | | | | |
| 29 (Ex) | C1-5 Alkyl | 5 | 10 | 37.5 | 37.5 | 10 | 99.5 ± 1.3 | — |
| | galactomannan | | | | | | | |

The compositions of the invention containing various oily gelling agents combined with Protium heptaphylum resin exhibit excellent dry friction resistance and good resistance in the presence of oil.

### Examples 30 to 38—Tack Test

### Protocol for Measuring the Tack

The support used is a strip of “Supplale”: synthetic leather sheet (polyurethane adhesively bonded to polyester-cotton fabric) which is white in colour and has the dimensions 150 mm×25 mm, sold under the name Supplale, code DFSUP15025B, supplier Soudotique). The product is deposited on the Supplale strip over 10 cm of length using an applicator so as to obtain a homogeneous deposit (4 passes of the applicator over the whole length, resubmerging the applicator in the product in between passes). The support with the sample is left to dry on a plate thermostatically controlled at 32° C. and ambient RH (50% RH) for 1 h. The specimen is next placed on the base of the texture analyser or on a metal plate provided with double-sided adhesive tape in order to hold it. The tack is measured using a TAXT2 plus texture analyser. The spindle (tip), 20 mm in diameter, containing a circle of fixed white Supplale®, is positioned on the texture analyser.

The parameters of the compression tests with hold over time are as follows: Approach speed (or pre-speed): 1 mm/s; Speed (starting from detection of contact): 0.5 mm/s; force: 800 g; hold time: 5 s; Return speed (or post-speed): 40 mm/s.

The tack is characterized by the work of detachment measured during the unloading (tensile phase), corresponding to the integral of the curve under the time axis. This work is expressed positively in joules per square metre. The lower the work value measured, the less tacky the deposit.

TABLE 9

| | | Ethyl- | | Isodo- | | Tack at |
|---|---|---|---|---|---|---|
| | Candelilla | cellulose, | Red7, | decane, | Ethanol, | 60 min, |
| Test | resin, wt % | wt % | wt % | wt % | wt % | J/m² |
| 30 (Ex) | 10.00 | 3.97 | 10.00 | 44.28 | 31.75 | 0.17 |
| 31 (Ex) | 20.00 | 3.97 | 10.00 | 36.28 | 29.75 | 0.19 |
| 32 (Ex) | 10.00 | 9.91 | 10.00 | 14.76 | 55.33 | 0.11 |
| 33 (Ex) | 20.00 | 9.91 | 10.00 | 6.76 | 53.33 | 0.11 |
| 34 (Ex) | 5.00 | 6.94 | 10.00 | 33.52 | 44.54 | 0.16 |
| 35 (Ex) | 25.00 | 6.94 | 10.00 | 17.52 | 40.54 | 0.12 |
| 36 (Ex) | 15.00 | 1.00 | 10.00 | 55.04 | 18.96 | 0.09 |
| 37 (Ex) | 15.00 | 6.94 | 10.00 | 25.52 | 42.54 | 0.11 |
| 38 (Ex) | 10.00 | 12.88 | 10.00 | 0.00 | 67.12 | 0.12 |

The limit of tack determined is 1.5 J/m². Beyond that, the deposit becomes perceptibly tacky.

All the deposits demonstrate a lack of tack after the films are dried (at 60 min).

The test results of the above table show that the film deposited from each of the compositions according to the invention does not have a tacky effect (0.12 J/m²).

### Examples 39 and 40—Other Examples of Compositions According to the Invention

The product is deposited using dip applicator (for the lips) on the forearm over an area of 3 cm×3 cm. The ease of spreading, the play time and the homogeneity are assessed qualitatively. The product is left drying until completely dry (around 5-10 min). The dry resistance is evaluated visually after contact with a tissue (intensity of mark). The resistance to olive oil is evaluated after adding a drop of olive oil on the deposit (spreading of oil manually over the entire surface of the deposit). Next, the resistance to olive oil is evaluated visually after contact with a tissue (intensity of mark).

TABLE 10

| | | % |
|---|---|---|
| INCI (US/EU) | Chemical name | Concentration |
| DENAT. ALCOHOL | DENATURED ABSOLUTE ETHYL ALCOHOL | 30.95 |
| ETHYLCELLULOSE | CELLULOSE, ETHYL ETHER | 13.4 |
| ISODODECANE | ISODODECANE | 20.65 |
| EUPHORBIA CERIFERA (CANDELILLA) | RESIN EXTRACTED FROM CANDELILLA | 20 |
| WAX EXTRACT/EUPHORBIA CERIFERA | WAX | |
| CERA EXTRACT | | |
| RED 7/CI 15850 | CALCIUM SALT OF LITHOL RUBINE B | 15 |

TABLE 11

| | | % |
|---|---|---|
| INCI (US/EU) | Chemical name | Concentration |
| ALCOHOL DENAT. | DENATURED ABSOLUTE ETHYL ALCOHOL | 34.26 |
| ETHYLCELLULOSE | CELLULOSE, ETHYL ETHER | 12.09 |
| ISODODECANE | ISODODECANE | 34.26 |
| EUPHORBIA CERIFERA (CANDELILLA) | RESIN EXTRACTED FROM CANDELILLA | 9.4. |
| WAX EXTRACT/EUPHORBIA CERIFERA | WAX | |
| CERA EXTRACT | | |
| RED 7/CI 15850 | CALCIUM SALT OF LITHOL RUBINE B | 10 |

### Examples 41 and 42—Results Obtained with Other Solvents

TABLE 12

| | | | | | | | [CR Fric | |
|---|---|---|---|---|---|---|---|---|
| | | Candelilla | Ethyl | Ethyl | | [CR Fric | Olive Oil/ | |
| | | Resin | cellulose | acetate | Pigments | Dry/CR | CR Dry] | Fragmentation |
| Test | | (wt %) | (wt %) | (wt %) | (wt %) | Dry] (%) | (%) | resistance |
| 41 | Ex | 5 | 5 | 80 | 10 | 95.4 ± | 96.7 ± | 1 |
| | | | | | | 5.9 | 1.7 | |

TABLE 13

| | | | | | | | | [CR Fric | |
|---|---|---|---|---|---|---|---|---|---|
| | | | | | | | [CR Fric | Olive | |
| | | Candelilla | Ethyl | Ethyl | Cetiol | | Dry/CR | Oil/CR | |
| | | Resin | cellulose | acetate | UT | Pigment | Dry] | Dry] | Fragmentation |
| Test | | (wt %) | (wt %) | (wt %) | (wt %) | (wt %) | (%) | (%) | resistance |
| 42 | Ex | 2 | 2 | 40 | 46 | 10 | 101.4 ± | 85.1 ± | 4 |
| | | | | | | | 1.1 | 6.6 | |

1. Cosmetic composition comprising, in a physiologically acceptable medium:
a—at least one volatile oil,
b—at least one volatile alcohol,
c—at least one natural resin, and
d—at least one modified polysaccharide.

2. Composition according to claim 1, in which a weight ratio of total amount of volatile oil(s) and of volatile alcohol(s) to total amount of natural resin(s) is greater than 1.

3. Composition according to claim 1, wherein the at least one volatile oil and/or the at least one volatile alcohol has a vapour pressure, at ambient temperature (25° C.) and atmospheric pressure, of greater than or equal to 1.3 Pa.

4. Composition according to claim 1, wherein the at least one volatile oil is chosen from volatile hydrocarbon-based oils, volatile silicone oils and mixtures thereof.

5. Composition according to claim 1, wherein the at least one volatile oil is at least partially of plant origin.

6. Composition according to claim 1, wherein a weight ratio of the amount of volatile oil(s) to the amount of natural resin(s) is from 0.5 to 50.

7. Composition according to claim 1, wherein the at least one volatile alcohol is chosen from C1-C4 alcohols.

8. Composition according to claim 1, wherein a weight ratio of the amount of volatile alcohol(s) to the amount of natural resin(s) is from 0.5 to 50.

9. Composition according to claim 1, wherein a weight ratio of the amount of volatile oil(s) to the amount of volatile alcohol(s) is from 0.01 to 100.

10. Composition according to claim 1, in which, in the composition, the weight content of the at least one volatile oil is greater than the weight content of the at least one volatile alcohol, which is greater than the weight content of the at least one natural resin, relative to the total weight of the composition.

11. Composition according to claim 1, in which said at least one resin is chosen from: a) acaroid resins, b) ambers, c) asphaltite and gilsonite, d) Peru balsam, e) Tolu balsam, f) benzoin resins, g) Canada balsam, h) copal resins, i) damars, j) elemis, k) frankincenses, l) galbanums, m) labdanums, n) mastics, o) myrrh, p) sandarac, q) shellacs, r) styrax, s) turpentine, t) rosins u) tall oils, v) resins extracted from plant waxes, and mixtures thereof.

12. Composition according to claim 1, wherein the at least one resin is selected from the group consisting of, the INCI name of which comprises at least one of the following terms: EUPHORBIA CERIFERA WAX EXTRACT, CANDELLILA WAX EXTRACT, PROTIUM HEPTAPHYILLUM RESIN, SHOREA ROBUSTA RESIN, GLYCERYL ROSINATE; and mixtures thereof.

13. Composition according to claim 1, wherein said at least one resin contains at least 30% of terpenic compounds by weight relative to the total weight of resin.

14. Composition according to claim 1, wherein said at least one resin comprises at least 10% by weight, of polyterpenic compounds relative to the total weight of resin.

15. Composition according to claim 1, wherein said at least one resin comprises less than 70% by weight of monoterpenic or sesquiterpenic compounds relative to the total weight of the resin.

16. Composition according to claim 1, wherein said at least one resin contains at least one diterpenic compound.

17. Composition according to claim 16, in which a total content of the at least one diterpenic compound of the resin is at least 20%; by weight of diterpenic compound(s) relative to the total weight of the natural resin.

18. Composition according to claim 1, wherein said at least one resin contains at least one triterpenic compound.

19. Composition according to claim 18, in which a total content of the at least one triterpenic compound is at least 10%, by weight of relative to the total weight of the resin.

20. Composition according to claim 1, wherein the at least one resin has a number-average molecular weight of less than or equal to 10 000 g/mol.

21. Composition according to claim 1, wherein said at least one resin has a glass transition temperature from 0° C. to 200° C.

22. Composition according to claim 1, wherein the at least one resin has a softening point from 20° C. to 150° C.

23. Composition according to claim 1, wherein the at least one resin is present in said composition at a content from 0.1% to 40% by weight relative to the total weight of the composition.

24. Composition according to claim 1, in which the at least one modified polysaccharide is chosen from cationic, nonionic, anionic or amphoteric polysaccharides.

25. Composition according to claim 1, in which the at least one modified polysaccharide is chosen from:
polysaccharide ethers which are alkyl polysaccharides, in which the alkyl radical comprises between 2 and 30;
polysaccharide esters; and mixtures thereof.

26. Composition according to claim 1, in which the at least one modified polysaccharide is present in the composition in an amount of 0.05% and 20% by weight with respect to the total weight of the composition, and/or
the weight ratio between the total amount of resin(s) and the total amount of modified polysaccharide(s) present in the composition, ranges is from 0.05 to 200.

27. Cosmetic composition comprising, in a physiologically acceptable medium, at least one oily phase having the composition of claim 1, said at least one oily phase being a continuous oily phase.

28. Composition according to claim 1, wherein the composition is in the form of an oily composition or of a water-in-oil emulsion or of a composition having several separate phases.

29. Composition according to claim 1, wherein the composition additionally has an aqueous phase at a total content by weight from 2 to 95% by weight relative to the total weight of said composition.

30. Composition according to claim 27, wherein the total content of at least one oily phase is from 5% to 100% by weight, relative to the total weight of the composition.

31. Composition according to claim 1, in which the at least one volatile oil is present at a content from 1% to 90% by weight relative to the total weight of the composition.

32. Composition according to claim 1, additionally comprising at least one pigment.

33. Composition according to claim 32, wherein the at least one pigment is present in a content of at least 2% by weight relative to the total weight of said composition.

34. Composition according to claim 1, in the form of a foundation, a lipstick, a mascara, an eyeliner, a concealer, corrector, an eyebrow product, a skincare product, a sun protection product, a hygiene product, a hair shaping product, a hair dyeing product, or a nail varnish.

35. Composition according to claim 1, wherein if the composition further comprises at least one synthetic resin, the composition comprises less than 10% by weight of synthetic resin relative to the total weight of the composition.

36. Composition according to claim 1, wherein if the composition further comprises at least one silicone oil, the composition comprises less than 30% by weight of silicone oil relative to the total weight of the composition.

37. Process for coating keratin materials comprising applying to the keratin materials a composition as defined according to claim 1.

38. A method of improving the persistence and/or the resistance to friction without increasing the tacky effect of a film obtained by applying a composition comprising applying a composition according to claim 1 to the keratin materials.
